(19) United States
(12) Patent Application Publication
(10) Pub. No.: US 2025/0064475 A1
(43) Pub. Date: Feb. 27, 2025

(54) PERICARDIAL TRANSECTION DEVICE WITH RETRACTABLE CUTTING APPARATUS

(71) Applicant: EDWARDS LIFESCIENCES CORPORATION, Irvine, CA (US)

(72) Inventors: Daniel James Murray, Orange, CA (US); Jose Islas, Corona, CA (US); Yaeer E. Lev, Torrance, CA (US)

(21) Appl. No.: 18/944,661

(22) Filed: Nov. 12, 2024

Related U.S. Application Data
(63) Continuation of application No. PCT/US2023/022931, filed on May 19, 2023.
(60) Provisional application No. 63/346,313, filed on May 26, 2022.

(51) Int. Cl. A61B 17/32; A61B 17/00; A61B 17/3211; A61M 25/00

(52) U.S. Cl. CPC A61B 17/320016; A61B 2017/00243; A61B 2017/32113; A61M 25/0082

(57) ABSTRACT

A medical device for creating elongated incisions within a pericardium is provided. The medical device includes an incision device operably coupled to a distal end of a catheter. The incision device includes an incision channel defined along the incision device. The medical device further includes a retractable cutting apparatus disposed within the incision channel. The retractable cutting apparatus includes a blade structured to move between a retracted position and a deployed position. In an instance in which the blade is in the retracted position, the blade is disposed within the incision channel. In an instance in which the blade is in the deployed position, the blade at least partially protrudes from the incision channel. The medical device further includes a cutting apparatus actuation mechanism structured to move the blade between the retracted position and the deployed position within a human body.

### RELATED APPLICATIONS

This application is a continuation application of International Patent Application No. PCT/US2023/022931, filed May 19, 2023, which claims the benefit of U.S. Provisional Application No. 63/346,313, filed on May 26, 2022. Each of the foregoing patent applications are hereby incorporated by reference in their entireties.

### TECHNICAL FIELD

This disclosure is directed to devices and methods for cutting in the pericardium. The devices and methods are generally applicable to the treatment of heart failure, for example, heart failure with preserved ejection fraction (HFpEF) or reduced injection fraction (HFrEF) by introducing one or more incision lengths in a pericardium, e.g., a parietal layer.

### BACKGROUND

Pericardial restraint is a normal physiologic process that becomes exaggerated, for example, in some patients with heart failure with preserved ejection fraction (HFpEF) and causes the right heart to run out of space when filling, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

In an example embodiment, a medical device for creating elongated incisions within a pericardium. The medical device includes a multi-lumen catheter including at least one lumen, a longitudinal axis, a proximal end, and a distal end. The medical device also includes an incision device operably coupled to the distal end of the multi-lumen catheter. The incision device includes an incision channel defined along a lateral side of catheter. The medical device also includes a retractable cutting apparatus disposed within the incision channel. The retractable cutting apparatus includes a blade configured to move between a retracted position and a deployed position. In an instance in which the blade is in the retracted position, the blade is disposed within the incision channel. In an instance in which the blade is in the deployed position, the blade at least partially protrudes from the incision channel. The medical device also includes a cutting apparatus actuation mechanism configured to move the blade between the retracted position and the deployed position within a human body.

In some example embodiments, upon movement of the blade into the deployed position, the retractable cutting apparatus is configured to be moved along the incision channel while the incision device remains stationary within the human. In some example embodiments, alone or in combination with any of the previous example embodiments, the blade of the cutting apparatus is configured to be moveable between the retracted position and the deployed position upon placement of the incision device within a pericardium of the human body.

In some example embodiments, alone or in combination with any of the previous example embodiments, the multi-lumen catheter is steerable. In some example embodiments, alone or in combination with any of the previous example embodiments, at least a portion of the multi-lumen catheter is radiopaque. In some example embodiments, alone or in combination with any of the previous example embodiments, the medical device also includes an introducer positioned near the distal end of the multi-lumen catheter. In some example embodiments, alone or in combination with any of the previous example embodiments, at least a portion of the introducer is radiopaque.

In some example embodiments, alone or in combination with any of the previous example embodiments, the at least one lumen comprises a guidewire lumen. In some example embodiments, alone or in combination with any of the previous example embodiments, the medical device includes a guidewire slidably positioned within the guidewire lumen. In some example embodiments, alone or in combination with any of the previous example embodiments, the blade includes a cutting surface positioned to cut tissue upon being moved within the human body in an instance in which the blade is in the deployed position.

In some example embodiments, alone or in combination with any of the previous example embodiments, the cutting surface of the blade when laterally projected, faces away from the distal end and towards the proximal end of the multi-lumen catheter. In some example embodiments, alone or in combination with any of the previous example embodiments, the medical device also includes one or more stabilizing members configured to maintain the multi-lumen catheter at a given location within the human body. In some example embodiments, alone or in combination with any of the previous example embodiments, the one or more stabilizing members are configured to move between a stabilizing retracted position and a stabilizing deployed position. In such embodiments, the one or more stabilizing members are moved from the stabilizing retracted position to the stabilizing deployed position upon positioning of the multi-lumen catheter within the human body.

In some example embodiments, alone or in combination with any of the previous example embodiments, the one or more stabilizing member is a wire, loop, or shape-memory metal. In some example embodiments, alone or in combination with any of the previous example embodiments, the one or more stabilizing member is one or more inflatable structures. In some example embodiments, alone or in combination with any of the previous example embodiments, the medical device includes a sheath configured to moveably cover the incision channel. In such an embodiment, the sheath is configured to cover the incision channel in an instance in which the multi-lumen catheter is not positioned within human body.

In some example embodiments, alone or in combination with any of the previous example embodiments, the one or more openings of the sheath are traversable along the multi-lumen catheter allowing an actuator to cause the one or more stabilizing members to laterally project through both the multi-lumen catheter and the sheath. In some example embodiments, alone or in combination with any of the previous example embodiments, the actuator concurrently or sequentially laterally projects the incision device and the one or more stabilizing members through the one or more openings of the multi-lumen catheter and the one or more openings of the sheath.

In some example embodiments, alone or in combination with any of the previous example embodiments, at least a portion of cutting surface is reversibly adjustable laterally relative to the longitudinal axis of the multi-lumen catheter between a range of angles. In some example embodiments, alone or in combination with any of the previous example embodiments, a plurality of radiopaque markers is provided along an edge of the incision channel and the position of the blade can be determined based on the radiopaque markers.

In some example embodiments, alone or in combination with any of the previous example embodiments, at least a portion of the cutting apparatus is radiopaque. In some example embodiments, alone or in combination with any of the previous example embodiments, the plurality of radiopaque markers is evenly spaced along the edge of the incision channel. In some example embodiments, alone or in combination with any of the previous example embodiments, the blade is configured to have a sufficient force to puncture pericardial tissue in an instance in which the blade is moved from the retracted position to the deployed position.

In some example embodiments, alone or in combination with any of the previous example embodiments, the medical device is sterilized. In some example embodiments, alone or in combination with any of the previous example embodiments, the medical device can be sterilized via gamma sterilization. In some example embodiments, alone or in combination with any of the previous example embodiments, the medical device can be sterilized via ethylene oxide sterilization. In some example embodiments, alone or in combination with any of the previous example embodiments, the medical device can be sterilized via autoclave sterilization. In some example embodiments, alone or in combination with any of the previous example embodiments, the medical device can be sterilized via E-beam sterilization. In some example embodiments, alone or in combination with any of the previous example embodiments, the incision device comprises a rigid housing and a cutting surface received by the rigid housing.

In an example embodiment, a medical device for creating elongated incisions within a pericardium is provided. The medical device includes a multi-lumen catheter including at least one lumen, a longitudinal axis, a proximal end, and a distal end. The medical device also includes an incision device operably coupled to the distal the multi-lumen catheter. The incision device includes an incision channel defined along the incision device. The medical device also includes a retractable cutting apparatus disposed within the incision channel. The retractable cutting apparatus includes a blade configured to move between a retracted position and a deployed position. The retractable cutting apparatus includes a translation body configured to be operably coupled to the blade. The retractable cutting apparatus also includes a blade rotation pin rotatably connecting the blade to the translation body. The blade rotates about the blade rotation pin. The retractable cutting apparatus includes an actuation pin attached to a cutting apparatus actuation mechanism configured to move the blade between the retracted position and the deployed position within a human body. The actuation pin moves along a blade slot defined on the blade and the movement of the actuation pin along the blade slot causes the blade to move between the retracted position and the deployed position. In an instance in which the blade is in the retracted position, the blade is disposed within the incision channel. In an instance in which the blade is in the deployed position, the blade at least partially protrudes from the incision channel.

In some example embodiments, alone or in combination with any of the previous example embodiments, upon movement of the blade into the deployed position, the retractable cutting apparatus is configured to be moved along the incision channel while the incision device remains stationary within the human. In some example embodiments, alone or in combination with any of the previous example embodiments, the blade of the cutting apparatus is configured to be moveable between the retracted position and the deployed position upon placement of the incision device within a pericardium of the human body. In some example embodiments, alone or in combination with any of the previous example embodiments, the blade of the cutting apparatus is curved. In some example embodiments, alone or in combination with any of the previous example embodiments, the blade of the cutting apparatus is curved in a direction of rotation from the retracted position to the deployed position.

In some example embodiments, alone or in combination with any of the previous example embodiments, the blade slot is curved. In some example embodiments, alone or in combination with any of the previous example embodiments, the medical device also includes a retractable cutting apparatus movement actuator attached to the translation body and the retractable cutting apparatus movement actuator is moved to move the retractable cutting apparatus along the incision channel. In some example embodiments, alone or in combination with any of the previous example embodiments, the retractable cutting apparatus movement actuator is actuated by a multi-lumen catheter controller.

In some example embodiments, alone or in combination with any of the previous example embodiments, the multi-lumen catheter is steerable. In some example embodiments, alone or in combination with any of the previous example embodiments, at least a portion of the multi-lumen catheter is radiopaque. In some example embodiments, alone or in combination with any of the previous example embodiments, the medical device includes an introducer positioned near the distal end of the multi-lumen catheter. In some example embodiments, alone or in combination with any of the previous example embodiments, at least a portion of the introducer is radiopaque. In some example embodiments, alone or in combination with any of the previous example embodiments, the at least one lumen comprises a guidewire lumen. In some example embodiments, alone or in combination with any of the previous example embodiments, the medical device includes a guidewire slidably positioned within the guidewire lumen.

In some example embodiments, alone or in combination with any of the previous example embodiments, the blade includes a cutting surface and the cutting surface is positioned to cut tissue upon being moved within the human body in an instance in which the blade is in the deployed position. In some example embodiments, alone or in combination with any of the previous example embodiments, the cutting surface of the blade when laterally projected, faces away from the distal end and towards the proximal end of the multi-lumen catheter. In some example embodiments, alone or in combination with any of the previous example embodiments, the medical device includes one or more stabilizing members configured to maintain the multi-lumen catheter at a given location within the human body. In some example embodiments, alone or in combination with any of the previous example embodiments, the one or more stabilizing members are configured to move between a stabilizing retracted position and a stabilizing deployed position, and the one or more stabilizing members are moved from the stabilizing retracted position to the stabilizing deployed position upon positioning of the multi-lumen catheter within the human body.

In some example embodiments, alone or in combination with any of the previous example embodiments, the one or more stabilizing member is a wire, loop, or shape-memory metal. In some example embodiments, alone or in combination with any of the previous example embodiments, the one or more stabilizing member is one or more inflatable structures. In some example embodiments, alone or in combination with any of the previous example embodiments, the medical device includes a sheath configured to moveably cover the incision channel, wherein the sheath is configured to cover the incision channel in an instance in which the multi-lumen catheter is not positioned within human body.

In some example embodiments, alone or in combination with any of the previous example embodiments, the one or more openings of the sheath are traversable along the multi-lumen catheter allowing an actuator to cause the one or more stabilizing members to laterally project through both the multi-lumen catheter and the sheath. In some example embodiments, alone or in combination with any of the previous example embodiments, the actuator concurrently or sequentially laterally projects the incision device and the one or more stabilizing members through the one or more openings of the multi-lumen catheter and the one or more openings of the sheath.

In some example embodiments, alone or in combination with any of the previous example embodiments, at least a portion of cutting surface is reversibly adjustable laterally relative to the longitudinal axis of the multi-lumen catheter between a range of angles. In some example embodiments, alone or in combination with any of the previous example embodiments, a plurality of radiopaque markers is provided along an edge of the incision channel and the position of the blade can be determined based on the radiopaque markers. In some example embodiments, alone or in combination with any of the previous example embodiments, at least a portion of the cutting apparatus is radiopaque.

In some example embodiments, alone or in combination with any of the previous example embodiments, the plurality of radiopaque markers is evenly spaced along the edge of the incision channel. In some example embodiments, alone or in combination with any of the previous example embodiments, the blade is configured to have a sufficient force to puncture pericardial tissue in an instance in which the blade is moved from the retracted position to the deployed position.

In some example embodiments, alone or in combination with any of the previous example embodiments, the medical device is sterilized. In some example embodiments, alone or in combination with any of the previous example embodiments, the medical device can be sterilized via gamma sterilization. In some example embodiments, alone or in combination with any of the previous example embodiments, the medical device can be sterilized via ethylene oxide sterilization. In some example embodiments, alone or in combination with any of the previous example embodiments, the medical device can be sterilized via autoclave sterilization. In some example embodiments, alone or in combination with any of the previous example embodiments, the medical device can be sterilized via E-beam sterilization.

In another example embodiment, a medical device for creating elongated incisions within a pericardium is provided. The medical device includes a multi-lumen catheter comprising at least one lumen, a longitudinal axis, a proximal end, and a distal end. The medical device also includes an incision device operably coupled to the distal the multi-lumen catheter. The incision device comprises an incision channel defined along the incision device. The medical device further includes a retractable cutting apparatus disposed within the incision channel. The retractable cutting apparatus includes a blade configured to move between a retracted position and a deployed position. The retractable cutting apparatus includes a translation body configured to be operably coupled to the blade and the translation body defines a translation body slot. The retractable cutting apparatus includes a blade movement pin attached to the blade, wherein the blade movement pin is positioned to move within the translation body slot. At least a first position within the translation body slot, the blade is in the retracted position and at a second position within the translation body slot, the blade is in the deployed position. The retractable cutting apparatus includes a cutting apparatus casing defining a translation body channel that allows the translation body to move within the translation body channel. The blade is rotatably attached to the cutting apparatus casing and the blade is configured to rotate based on the movement of the translation body. Upon moving the translation body in a first direction, the blade movement pin moves along the translation body slot causing the blade to rotate from a clockwise direction in the retracted position to the deployed position. In an instance in which the blade is in the retracted position, the blade is disposed within the incision channel. In an instance in which the blade is in the deployed position, the blade at least partially protrudes from the incision channel.

In some example embodiments, alone or in combination with any of the previous example embodiments, upon moving the translation body in a second direction opposite the first direction, the blade movement pin moves along the translation body slot causing the blade to rotate in a counterclockwise direction from the deployed position to the retracted position. In some example embodiments, alone or in combination with any of the previous example embodiments, upon movement of the blade into the deployed position, the retractable cutting apparatus is configured to be moved along the incision channel while the incision device remains stationary within the human. In some example embodiments, alone or in combination with any of the previous example embodiments, the blade of the cutting apparatus is configured to be moveable between the retracted position and the deployed position upon placement of the incision device within a pericardium of the human body.

In some example embodiments, alone or in combination with any of the previous example embodiments, the cutting apparatus casing defines at least one guidewire channel configured to receive a guidewire and the cutting apparatus casing travels along the guidewire upon movement of the retractable cutting apparatus. In some example embodiments, alone or in combination with any of the previous example embodiments, the medical device includes a retractable cutting apparatus movement actuator attached to the cutting apparatus casing and the retractable cutting apparatus movement actuator is moved to move the retractable cutting apparatus along the incision channel. In some example embodiments, alone or in combination with any of the previous example embodiments, the retractable cutting apparatus movement actuator is actuated by a multi-lumen catheter controller. In some example embodiments, alone or in combination with any of the previous example embodiments, the multi-lumen catheter is steerable. The medical device of any of the preceding claims, wherein at least a portion of the multi-lumen catheter is radiopaque.

In some example embodiments, alone or in combination with any of the previous example embodiments, the medical device includes an introducer positioned near the distal end of the multi-lumen catheter. In some example embodiments, alone or in combination with any of the previous example embodiments, a portion of the introducer is radiopaque. In some example embodiments, alone or in combination with any of the previous example embodiments, the at least one lumen comprises a guidewire lumen. In some example embodiments, alone or in combination with any of the previous example embodiments, the medical device also includes a guidewire slidably positioned within the guidewire lumen.

In some example embodiments, alone or in combination with any of the previous example embodiments, the blade includes a cutting surface positioned to cut tissue upon being moved within the human body in an instance in which the blade is in the deployed position. In some example embodiments, alone or in combination with any of the previous example embodiments, the cutting surface of the blade when laterally projected, faces away from the distal end and towards the proximal end of the multi-lumen catheter. In some example embodiments, alone or in combination with any of the previous example embodiments, the medical device includes one or more stabilizing members configured to maintain the multi-lumen catheter at a given location within the human body.

In some example embodiments, alone or in combination with any of the previous example embodiments, the one or more stabilizing members are configured to move between a stabilizing retracted position and a stabilizing deployed position, and the one or more stabilizing members are moved from the stabilizing retracted position to the stabilizing deployed position upon positioning of the multi-lumen catheter within the human body.

In some example embodiments, alone or in combination with any of the previous example embodiments, the one or more stabilizing member is a wire, loop, or shape-memory metal. In some example embodiments, alone or in combination with any of the previous example embodiments, the one or more stabilizing member is one or more inflatable structures. In some example embodiments, alone or in combination with any of the previous example embodiments, the medical device includes a sheath configured to moveably cover the incision channel and the sheath is configured to cover the incision channel in an instance in which the multi-lumen catheter is not positioned within human body.

In some example embodiments, alone or in combination with any of the previous example embodiments, the one or more openings of the sheath are traversable along the multi-lumen catheter allowing an actuator to cause the one or more stabilizing members to laterally project through both the multi-lumen catheter and the sheath. In some example embodiments, alone or in combination with any of the previous example embodiments, the actuator concurrently or sequentially laterally projects the incision device and the one or more stabilizing members through the one or more openings of the multi-lumen catheter and the one or more openings of the sheath. In some example embodiments, alone or in combination with any of the previous example embodiments, at least a portion of cutting surface is reversibly adjustable laterally relative to the longitudinal axis of the multi-lumen catheter between a range of angles.

In some example embodiments, alone or in combination with any of the previous example embodiments, a plurality of radiopaque markers is provided along an edge of the incision channel and the position of the blade can be determined based on the radiopaque markers. In some example embodiments, alone or in combination with any of the previous example embodiments, at least a portion of the cutting apparatus is radiopaque. In some example embodiments, alone or in combination with any of the previous example embodiments, the plurality of radiopaque markers is evenly spaced along the edge of the incision channel. In some example embodiments, alone or in combination with any of the previous example embodiments, the blade is configured to have a sufficient force to puncture pericardial tissue in an instance in which the blade is moved from the retracted position to the deployed position.

In some example embodiments, alone or in combination with any of the previous example embodiments, the medical device is sterilized. In some example embodiments, alone or in combination with any of the previous example embodiments, the medical device can be sterilized via gamma sterilization. In some example embodiments, alone or in combination with any of the previous example embodiments, the medical device can be sterilized via ethylene oxide sterilization. In some example embodiments, alone or in combination with any of the previous example embodiments, the medical device can be sterilized via autoclave sterilization. In some example embodiments, alone or in combination with any of the previous example embodiments, the medical device can be sterilized via E-beam sterilization.

In still another example embodiment, a medical device for creating elongated incisions within a pericardium. The medical device includes a multi-lumen catheter including at least one lumen, a longitudinal axis, a proximal end, and a distal end. The medical device includes an incision device operably coupled to the distal the multi-lumen catheter. The incision device includes an incision channel defined along the incision device. The medical device includes a retractable cutting apparatus disposed within the incision channel. The retractable cutting apparatus includes a blade configured to move between a retracted position and a deployed position. The medical device includes a cutting apparatus actuation mechanism attached to the blade. The cutting apparatus actuation mechanism is configured to move the blade between the retracted position and the deployed position within a human body. The incision channel defines a first retracted area defined at a first end of the incision channel, a deployed area, and a second retracted area defined at a second end of the incision channel opposite the first end. The blade upon actuation by the cutting apparatus actuation mechanism moves from the retracted position within the first retracted area into the deployed position within the deployed area and returns to the retracted position within the second retracted position. In an instance in which the blade is in the retracted position, the blade is disposed within the incision channel. In an instance in which the blade is in the deployed position, the blade at least partially protrudes from the incision channel.

In some example embodiments, alone or in combination with any of the previous example embodiments, upon movement of the blade into the deployed position, the retractable cutting apparatus is configured to be moved along the incision channel while the incision device remains stationary within the human. In some example embodiments, alone or in combination with any of the previous example embodiments, the blade of the cutting apparatus is configured to be moveable between the retracted position and the deployed position upon placement of the incision device within a pericardium of the human body.

In some example embodiments, alone or in combination with any of the previous example embodiments, the medical device includes a deployment ramp defined between the first retracted area and the deployed area and the blade is caused to rotate from the retracted position to the deployed position upon engaging the deployment ramp. In some example embodiments, alone or in combination with any of the previous example embodiments, the medical device includes a retraction ramp defined between the deployed area and the second retracted area and the blade is caused to rotate from the deployed position to the retracted position upon engaging the deployment ramp.

In some example embodiments, alone or in combination with any of the previous example embodiments, the multi-lumen catheter is steerable. In some example embodiments, alone or in combination with any of the previous example embodiments, at least a portion of the multi-lumen catheter is radiopaque. In some example embodiments, alone or in combination with any of the previous example embodiments, the medical device includes an introducer positioned near the distal end of the multi-lumen catheter. In some example embodiments, alone or in combination with any of the previous example embodiments, at least a portion of the introducer is radiopaque.

In some example embodiments, alone or in combination with any of the previous example embodiments, the at least one lumen includes a guidewire lumen. In some example embodiments, alone or in combination with any of the previous example embodiments, the medical device includes a guidewire slidably positioned within the guidewire lumen. In some example embodiments, alone or in combination with any of the previous example embodiments, the blade includes a cutting surface and the cutting surface is positioned to cut tissue upon being moved within the human body in an instance in which the blade is in the deployed position. In some example embodiments, alone or in combination with any of the previous example embodiments, the cutting surface of the blade when laterally projected, faces away from the distal end and towards the proximal end of the multi-lumen catheter.

In some example embodiments, alone or in combination with any of the previous example embodiments, the medical device includes one or more stabilizing members configured to maintain the multi-lumen catheter at a given location within the human body. In some example embodiments, alone or in combination with any of the previous example embodiments, the one or more stabilizing members are configured to move between a stabilizing retracted position and a stabilizing deployed position, and the one or more stabilizing members are moved from the stabilizing retracted position to the stabilizing deployed position upon positioning of the multi-lumen catheter within the human body.

In some example embodiments, alone or in combination with any of the previous example embodiments, the one or more stabilizing member is a wire, loop, or shape-memory metal. In some example embodiments, alone or in combination with any of the previous example embodiments, the one or more stabilizing member is one or more inflatable structures. In some example embodiments, alone or in combination with any of the previous example embodiments, the medical device includes a sheath configured to moveably cover the incision channel and the sheath is configured to cover the incision channel in an instance in which the multi-lumen catheter is not positioned within human body.

In some example embodiments, alone or in combination with any of the previous example embodiments, the one or more openings of the sheath are traversable along the multi-lumen catheter allowing an actuator to cause the one or more stabilizing members to laterally project through both the multi-lumen catheter and the sheath. In some example embodiments, alone or in combination with any of the previous example embodiments, the actuator concurrently or sequentially laterally projects the incision device and the one or more stabilizing members through the one or more openings of the multi-lumen catheter and the one or more openings of the sheath.

In some example embodiments, alone or in combination with any of the previous example embodiments, at least a portion of cutting surface is reversibly adjustable laterally relative to the longitudinal axis of the multi-lumen catheter between a range of angles. In some example embodiments, alone or in combination with any of the previous example embodiments, a plurality of radiopaque markers is provided along an edge of the incision channel and the position of the blade can be determined based on the radiopaque markers. In some example embodiments, alone or in combination with any of the previous example embodiments, at least a portion of the cutting apparatus is radiopaque.

In some example embodiments, alone or in combination with any of the previous example embodiments, the plurality of radiopaque markers is evenly spaced along the edge of the incision channel. In some example embodiments, alone or in combination with any of the previous example embodiments, the blade is configured to have a sufficient force to puncture pericardial tissue in an instance in which the blade is moved from the retracted position to the deployed position.

In some example embodiments, alone or in combination with any of the previous example embodiments, the medical device is sterilized. In some example embodiments, alone or in combination with any of the previous example embodiments, the medical device can be sterilized via gamma sterilization. In some example embodiments, alone or in combination with any of the previous example embodiments, the medical device can be sterilized via ethylene oxide sterilization. In some example embodiments, alone or in combination with any of the previous example embodiments, the medical device can be sterilized via autoclave sterilization. In some example embodiments, alone or in combination with any of the previous example embodiments, the medical device can be sterilized via E-beam sterilization.

In another example embodiment, a method of creating elongated incisions within a pericardium. The method includes placing a multi-lumen catheter within a human. The multi-lumen catheter includes at least one lumen, a longitudinal axis, a proximal end, and a distal end. The method also includes positioning an incision device within the pericardium. The incision device is operably coupled to the distal end of the multi-lumen catheter. The incision device includes an incision channel defined along the incision device. The method includes moving a blade of a retractable cutting apparatus disposed within the incision channel from a retracted position to a deployed position. In an instance in which the blade is in the retracted position, the blade is disposed within the incision channel. In an instance in which the blade is in the deployed position, the blade at least partially protrudes from the incision channel. The method includes moving the retractable cutting apparatus along the incision channel while the incision device remains stationary within the pericardium. The blade moves in the direction of the proximal end of the multi-lumen catheter.

In some example embodiments, alone or in combination with any of the previous example embodiments, the retractable cutting apparatus along the incision channel while the incision device remains stationary within the pericardium causes the blade to create at least one incision length through the pericardium. In some example embodiments, alone or in combination with any of the previous example embodiments, the creation of the at least one incision length through the pericardium causes a reduction in pressure exerted on a heart by the pericardium. In some example embodiments, alone or in combination with any of the previous example embodiments, the incision length is created in at least one of a parietal layer or fibrous layer of the pericardium. In some example embodiments, alone or in combination with any of the previous example embodiments, the incision length is created in adipose tissue or fat deposits. In some example embodiments, alone or in combination with any of the previous example embodiments, the at least one incision length is along a length or circumference of only the parietal layer of the pericardium. In some example embodiments, alone or in combination with any of the previous example embodiments, the at least one incision length is made in the parietal layer from an anterior to a posterior of a heart. In some example embodiments, alone or in combination with any of the previous example embodiments, the at least one incision length is made in the parietal layer from a posterior base to an apex of a heart. In some example embodiments, alone or in combination with any of the previous example embodiments, the at least one incision length is made in the parietal layer from a posterior right atrium to an apex of a heart.

In some example embodiments, alone or in combination with any of the previous example embodiments, the at least one incision length is made in the parietal layer from a left ascending aorta to an apex of a heart. In some example embodiments, alone or in combination with any of the previous example embodiments, the at least one incision length is made in the parietal layer from a right ascending aorta to an apex of a heart. In some example embodiments, alone or in combination with any of the previous example embodiments, the at least one incision length is made in the parietal layer transversely about a heart.

In some example embodiments, alone or in combination with any of the previous example embodiments, the method includes, before creating the incision length, puncturing pericardial tissue and providing an access point into a pericardial space. In some example embodiments, alone or in combination with any of the previous example embodiments, the method includes, before puncturing, providing subxiphoid access to the pericardium. In some example embodiments, alone or in combination with any of the previous example embodiments, the method includes, before puncturing, providing transvascular access to the pericardium. In some example embodiments, alone or in combination with any of the previous example embodiments, the method includes moving the blade from the deployed position to the retracted position before removing the incision device from the pericardium. In some example embodiments, alone or in combination with any of the previous example embodiments, the method includes puncturing into the pericardium via an introducer.

In some example embodiments, alone or in combination with any of the previous example embodiments, the method includes, after puncturing, inserting a guidewire into the pericardial space. In some example embodiments, alone or in combination with any of the previous example embodiments, the method includes stabilizing the incision device within the pericardium. In some example embodiments, alone or in combination with any of the previous example embodiments, the incision device is stabilized via one or more stabilization member. In some example embodiments, alone or in combination with any of the previous example embodiments, the method includes, the one or more stabilizing members are configured to move between a stabilizing retracted position and a stabilizing deployed position, and the one or more stabilizing members are moved from the stabilizing retracted position to the stabilizing deployed position upon positioning of the multi-lumen catheter within the human body. In some example embodiments, alone or in combination with any of the previous example embodiments, the one or more stabilizing member is a wire, loop, or shape-memory metal. In some example embodiments, alone or in combination with any of the previous example embodiments, the one or more stabilizing member is one or more inflatable structures.

In some example embodiments, alone or in combination with any of the previous example embodiments, the method includes moving a sheath from a first position covering the incision channel to a second position that uncovers the incision channel. In some example embodiments, alone or in combination with any of the previous example embodiments, the method includes the sheath is configured to cover the incision channel in an instance in which the multi-lumen catheter is not positioned within human body. In some example embodiments, alone or in combination with any of the previous example embodiments, the method includes the one or more openings of the sheath are traversable along the multi-lumen catheter allowing an actuator to cause the one or more stabilizing members to laterally project through both the multi-lumen catheter and the sheath.

In some example embodiments, alone or in combination with any of the previous example embodiments, the method includes sterilizing the incision device. In some example embodiments, alone or in combination with any of the previous example embodiments, the incision device is sterilized via E-beam sterilization. In some example embodiments, alone or in combination with any of the previous example embodiments, the incision device is sterilized via gamma sterilization. In some example embodiments, alone or in combination with any of the previous example embodiments, the incision device is sterilized via ethylene oxide sterilization. In some example embodiments, alone or in combination with any of the previous example embodiments, the incision device is sterilized via autoclave sterilization.

In some example embodiments, alone or in combination with any of the previous example embodiments, the method includes determining a location of the incision device within the pericardium via ultrasound. In some example embodiments, alone or in combination with any of the previous example embodiments, the method includes creating the at least one incision length is determined in response to a signal indicative of a reduction of restraint of the heart. In some example embodiments, alone or in combination with any of the previous example embodiments, the method includes creating the at least one incision length is determined in response to a signal indicative of a reduction of restraint of the heart; and repeating the creating of the at least one incision length.

In some example embodiments, alone or in combination with any of the previous example embodiments, the method includes, after creating the at least one incision length, confirming a location of a distal end of the multi-lumen catheter device; and in response to a signal indicative of a reduction of restraint of the heart, repeating the steps of creating the at least one incision length, and confirming a location of the distal end.

In another example, a medical device for creating elongated incisions within a pericardium is provided. The medical device includes an incision device configured to be operably coupled to a distal end of a catheter. The incision device includes an incision channel defined along the incision device. The medical device also includes a retractable cutting apparatus disposed within the incision channel. The retractable cutting apparatus includes a blade configured to move between a retracted position and a deployed position. The retractable cutting apparatus includes a translation body configured to be operably coupled to the blade. The retractable cutting apparatus also includes a blade rotation pin rotatably connecting the blade to the translation body with the blade rotating about the blade rotation pin. The retractable cutting apparatus further includes an actuation pin attached to a cutting apparatus actuation mechanism configured to move the blade between the retracted position and the deployed position. The actuation pin moves along a blade slot defined on the blade and the movement of the actuation pin along the blade slot causes the blade to move between the retracted position and the deployed position. In an instance in which the blade is in the retracted position, the blade is disposed within the incision channel. In an instance in which the blade is in the deployed position, the blade at least partially protrudes from the incision channel.

In one aspect, alone or in combination with any of the previous aspects, upon movement of the blade into the deployed position, the retractable cutting apparatus is configured to be moved along the incision channel while the incision device remains stationary within a human body.

In one aspect, alone or in combination with any of the previous aspects, the blade of the cutting apparatus is curved.

In one aspect, alone or in combination with any of the previous aspects, the blade of the cutting apparatus is curved in a direction of rotation from the retracted position to the deployed position.

In one aspect, alone or in combination with any of the previous aspects, the blade slot is curved.

In one aspect, alone or in combination with any of the previous aspects, the medical device also includes a retractable cutting apparatus movement actuator attached to the translation body and the retractable cutting apparatus movement actuator is movable to move the retractable cutting apparatus along the incision channel.

In one aspect, alone or in combination with any of the previous aspects, the retractable cutting apparatus movement actuator is actuated by a catheter controller.

In one aspect, alone or in combination with any of the previous aspects, the medical device also includes an introducer positioned near a distal end of the incision device.

In one aspect, alone or in combination with any of the previous aspects, at least a portion of the introducer is radiopaque.

In one aspect, alone or in combination with any of the previous aspects, a cutting surface of the blade when laterally projected, faces away from a distal end and towards a proximal end of the catheter.

In one aspect, alone or in combination with any of the previous aspects, the medical device also includes one or more stabilizing members configured to maintain the catheter at a given location within a human body.

In one aspect, alone or in combination with any of the previous aspects, the one or more stabilizing members are configured to move between a stabilizing retracted position and a stabilizing deployed position with the one or more stabilizing members being moved from the stabilizing retracted position to the stabilizing deployed position upon positioning of the multi-lumen catheter within the human body.

In one aspect, alone or in combination with any of the previous aspects, at least a portion of cutting surface is reversibly adjustable laterally relative to a longitudinal axis of the catheter between a range of angles.

In one aspect, alone or in combination with any of the previous aspects, a plurality of radiopaque markers is provided along an edge of the incision channel, wherein the position of the blade can be determined based on the radiopaque markers.

In one aspect, alone or in combination with any of the previous aspects, the plurality of radiopaque markers is evenly spaced along the edge of the incision channel.

In one aspect, alone or in combination with any of the previous aspects, the medical device is sterilized.

In another example, a medical device for creating elongated incisions within a pericardium is provided. The medical device includes an incision device configured to be operably coupled to a distal end of a catheter with the incision device including an incision channel defined along the incision device. The medical device also includes a retractable cutting apparatus disposed within the incision channel. The retractable cutting apparatus includes a blade configured to move between a retracted position and a deployed position. The retractable cutting apparatus includes a translation body configured to be operably coupled to the blade. The translation body defines a translation body slot. The retractable cutting apparatus also includes a blade movement pin attached to the blade with the blade movement pin being positioned to move within the translation body slot. At a first position within the translation body slot, the blade is in the retracted position and at a second position within the translation body slot, the blade is in the deployed position. The retractable cutting apparatus further includes a cutting apparatus casing defining a translation body channel that allows the translation body to move within the translation body channel. The blade is rotatably attached to the cutting apparatus casing and the blade is configured to rotate based on the movement of the translation body. Upon moving the translation body in a first direction, the blade movement pin moves along the translation body slot causing the blade to rotate from a clockwise direction in the retracted position to the deployed position. In an instance in which the blade is in the retracted position, the blade is disposed within the incision channel. In an instance in which the blade is in the deployed position, the blade at least partially protrudes from the incision channel.

In one aspect, alone or in combination with any of the previous aspects, the medical device includes a controller engaged to the incision device.

In one aspect, alone or in combination with any of the previous aspects, the controller is configured to provide at least one of movement of the blade between the retracted position and the deployed position, movement of the retractable cutting apparatus along the incision device, energy to the incision device, or movement of one or more stabilizing members.

In another example embodiment, a method of manipulating a medical device is provided. The method includes providing a medical device of any of the embodiments disclosed herein engage with a controller; and controlling at least one of movement of the blade between the retracted position and the deployed position, movement of the retractable cutting apparatus along the incision device, energy to the incision device, or movement of one or more stabilizing members.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIG. 1C is a further enlarged view of section 1C of FIG. 1A depicting the serosal, visceral, fibrous layers and adipose tissue of the parietal pericardium, including the pericardial cavity.

FIG. 2 is a medical device of an example embodiment in the retracted position.

FIGS. 3A-3D is various views of a retractable cutting apparatus in accordance with various embodiments.

FIG. 4A is another example retractable cutting apparatus in the deployed position in accordance with various embodiments.

FIG. 4B is a side view of the retractable cutting apparatus shown in FIG. 4A in the retracted position in accordance with various embodiments.

FIGS. 5A and 5B are exploded views of a medical device with the example retractable cutting apparatus shown in FIGS. 4A and 4B in accordance with various embodiments

FIGS. 6A-6B are various exploded views of the example retractable cutting apparatus shown in FIGS. 4A-5B in accordance with various embodiments.

FIG. 7A is a medical device of an example embodiment with another example retractable cutting apparatus in the retracted position in accordance with various embodiments.

FIG. 7B is a medical device of an example embodiment with the example retractable cutting apparatus in the deployed position in accordance with various embodiments.

FIG. 7C is a medical device of an example embodiment with the example retractable cutting apparatus in the retracted position in accordance with various embodiments.

FIG. 8 is a medical device with stabilizing member of an example embodiment with the example retractable cutting apparatus in the deployed position in accordance with various embodiments.

FIG. 9A is an example controller for delivering and/or operating the incision devices in accordance with various embodiments.

FIG. 9B is another example controller for delivering and/or operating the incision devices in accordance with various embodiments.

FIG. 9C is a cross-section of the example controller shown in FIG. 9B in accordance with various embodiments.

FIG. 10A illustrates an example medical device positioned within the pericardial cavity in accordance with various embodiments.

FIG. 10B illustrates a blade of an example retractable cutting apparatus puncturing a parietal layer of the pericardium.

FIG. 10C illustrates a blade of an example retractable cutting apparatus making the incision along the parietal layer of the pericardium.

FIG. 11 is an example retractable cutting apparatus connected to an electrical current in accordance with various embodiments.

FIG. 12 is a simplified diagram of a transcatheter approach to the pericardial cavity in accordance with various embodiments.

FIG. 13 is a simplified diagram of an alternative transcatheter approach to the pericardial cavity in accordance with various embodiments.

FIG. 14 is a simplified diagram of a parietal layer incision length and cut path in accordance with various embodiments.

### DETAILED DESCRIPTION

The present disclosure provides for a catheter-based therapy referred to as transcatheter alleviation of pericardial restraint (TAPR) that can reduce pericardial restraint by incising or opening the pericardium with the intention of improving patient outcomes with heart dysfunction, for example, HFpEF or HFrEF and reducing HF readmissions related thereto. The present disclosure, in one example, provides a device with a concealed/medially-facing cutting surface for accessing and modifying a subject's pericardium for relieving pericardial restraint and/or resolving a heart dysfunction. The present disclosure further provides for methods of treating heart dysfunction using the presently disclosed device.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

As used herein the phrase “pericardial restraint” includes any terms that signify impaired filling caused by excessive forces from the pericardium.

As used herein the phrase “cutting surface” is inclusive of one or more of an edge of a sharpened blade or the surface of an electrode configured to receive sufficient current or radio frequency energy (RF) to ablate, burn, vaporize, or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a multi-lumen catheter device or catheter, and the application of a directional force sufficient to cut or separate the tissue, the force being substantially in a direction towards the proximal end of the multi-lumen catheter device or catheter, for example, by pulling the multi-lumen catheter device or catheter while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and the like.

As used herein the phrase “incision length” is inclusive of a non-zero distance of a cut or incision, for example, beginning at a first point, e.g., a target point, and terminating at a second point, e.g., an access point. An incision length can be linear, non-linear, or a plurality of linear and/or non-linear lengths that intersect or do not intersect about a curved or non-planar surface, such a heart.

As used herein the phrase “reducing pressure” and “reducing restraint” are inclusive of their ordinary and customary meaning of one to ordinary skill in medical and surgical arts.

As used herein the phrase “reduced ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “reduced ejection fraction” is inclusive of the ordinary and customary meaning to one of ordinary skill in medical and surgical arts, for example, impairment of ventricular filling or ejection of blood or both, with a clinical syndrome in which patients display left ventricular ejection fraction (LVEF) of 40% or less and are accompanied by progressive left ventricular dilatation and adverse cardiac remodeling and/or mitral valve dysfunction.

As used herein the phrase “heart dysfunction” is inclusive of the ordinary and customary meaning to one of ordinary skill in medical and surgical arts, for example, heart failure or congestive heart failure.

As used herein the phrase “incision device” is inclusive of a device with a cutting surface, for example an edge of a blade or a surface of an electrode.

As used herein the phrase “pericardial incision assembly” and “incision assembly” are used interchangeable and refer to an assemblage that includes an incision device.

As used herein the phrase “multi-lumen catheter device” is inclusive of a catheter configured with at least one lumen comprising a medical instrument, device, or component thereof, for example, an incision device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein the phrase “puncturing tip” is inclusive of an atraumatic object suitable for puncturing or penetrating tissue without substantial trauma to or bleeding from the vicinity of the picture or penetration.

Several exemplary transection devices for making incisions through the pericardial membrane or parietal layer of the pericardium are described herein. These examples share the characteristic that they may be delivered intravascularly through the right atrial appendage (RAA), Inferior Vena Cava (IVC), Superior Vena Cava (SVC), Coronary Sinus (CS), Right Atrium (RA), Left Atrial Appendage (LAA), or Right Ventricle (RV). Alternatively, devices may be delivered to the pericardial cavity through catheter access via a subxiphoid approach.

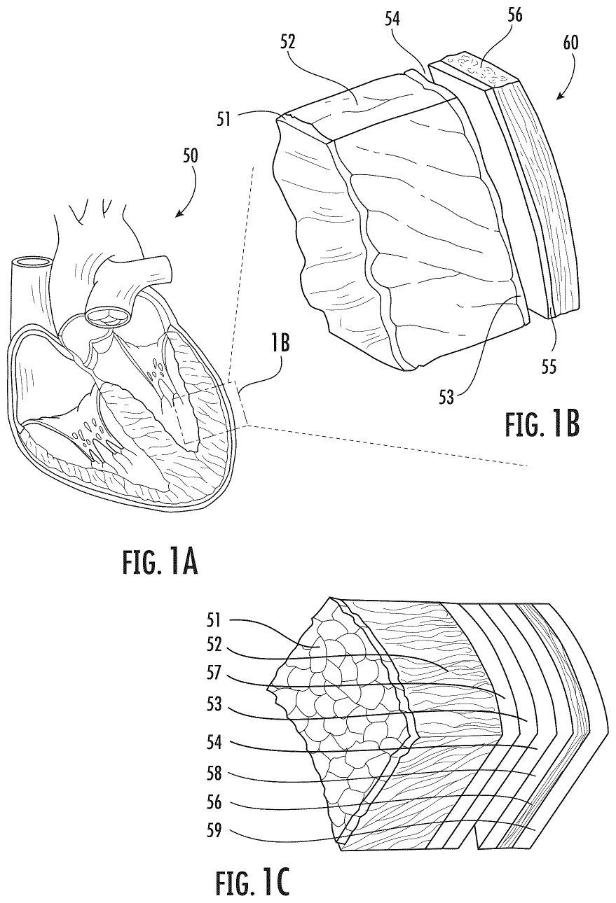

With reference to FIGS. 1A, 1B, 1C, and section 1B, layers of a heart wall of a heart 50, with pericardium 60, from inside-out, being the endocardium 51, the myocardium 52, epicardial adipose tissue 57, the visceral layer 53, the pericardial cavity 54, the parietal layer 55, the fibrous pericardium 56, and the pericardial adipose tissue 59 are depicted. In one example, the presently disclosed devices are configured for introduction to the pericardial cavity 54 and for cutting tissue layers generally disposed adjacent to and including adipose tissue within and outside the pericardial cavity and superficial to the visceral layer 53 of the pericardium 60.

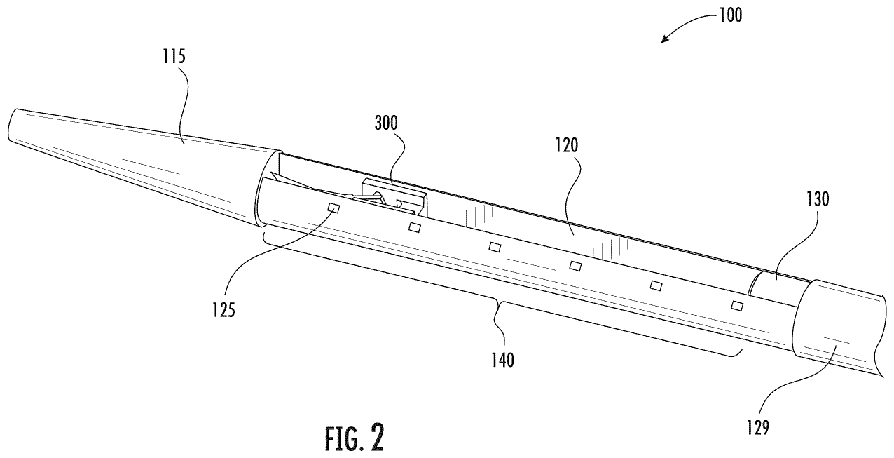

Referring now to FIG. 2, in one example the presently disclosed medical device 100 comprises an elongate catheter 129 comprising a distal end, at least one lumen, and a longitudinal axis; an incision device 140 coupled to the distal end of the catheter; and an introducer 115 coupled to and projecting from the incision device 140 In one example, the introducer 115 tapers to be flush with catheter 129. In one example, at least a portion of the catheter 129 tip is radiopaque. In one example, at least a portion of the incision assembly tip (e.g., the sharp edge of blade 305) is radiopaque. In one example, at least a portion of the introducer 115 tip is radiopaque. The catheter 129 can be relatively flexible to follow along the pericardial cavity or alternatively relatively stiff.

The incision device 140 defines an incision channel 120 that is configured to receive a retractable cutting apparatus 300. The incision channel 120 can extend along a lateral side of the catheter 129. The incision channel 120 can be aligned along or parallel with a longitudinal axis of the catheter 129. While retractable cutting apparatus 300 is shown in FIG. 2, various other retractable cutting apparatuses discussed herein can also be disposed within the incision channel (e.g., retractable cutting apparatus 400 or retractable cutting apparatus 700). The retractable cutting apparatus can be moved along the incision channel via an internal actuator (e.g., cutting apparatus movement actuator 130).

FIG. 2 illustrates the blade of the retractable cutting apparatus 300 in the retracted position. As discussed herein, the blade is configured to move between a retracted position and a deployed position. In the retracted position, the blade is disposed within the incision channel (e.g., not protruding from the incision channel). In the deployed position, the blade at least partially protrudes from the incision channel.

As shown, the incision device 140 may have one or more markers 125 that are positioned adjacent to the incision channel. The markers 125 may be radiopaque or otherwise viewable by an ultrasound. The retractable cutting apparatus 300 may also be at least partially radiopaque. The markers 125 may be spaced along the incision channel. The spacing of the markers 125 along the incision channel 120 may be the same from one marker to the next marker. As such, the location of the retractable cutting apparatus within the incision channel while the incision device is disposed within the pericardium can be determined using the markers 125.

Part or all of the medical device may be sterilized for use. The medical device may be sterilized using various sterilizing techniques, such as E-Beam sterilization, gamma sterilization, ethylene oxide sterilization, autoclave sterilization, and/or the like. Additionally, one or more materials used in the medical device may have anti-bacterial characteristics.

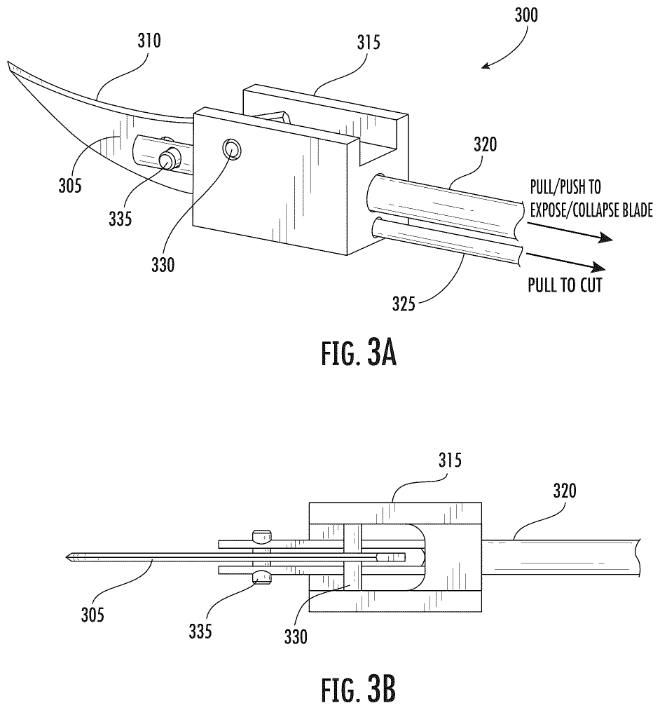

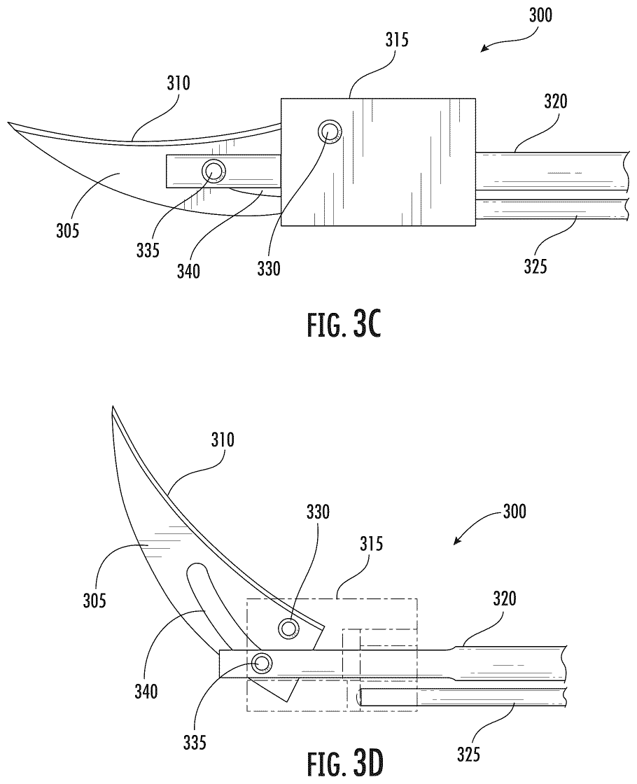

Referring now to FIGS. 3A-3D, an example retractable cutting apparatus 300 is shown. As discussed above in reference to FIG. 2, the retractable cutting apparatus 300 is movably positioned within the incision channel 120 of the incision device. The retractable cutting apparatus 300 includes a translation body 315, a blade 305, a cutting apparatus actuation mechanism 320, and a retractable cutting apparatus movement actuator 325.

FIGS. 3A-3C each illustrate the blade 305 of the retractable cutting apparatus 300 in the retracted position. FIG. 3D illustrates the blade 305 of the retractable cutting apparatus 300 in the retracted position. The blade 305 is rotatably attached to the translation body 315 via a blade rotation pin 330. The blade 305 rotates about the blade rotation pin 330 during movement between the retracted position and the deployed position.

The blade 305 has a blade slot 340 that is configured to receive an actuation pin 335. The actuation pin 335 is attached to the cutting apparatus actuation mechanism 320. The actuation pin 335 moves based within the blade slot 340 in response to the actuation of the cutting apparatus actuation mechanism 320. As the actuation pin 335 moves, the blade 305 is caused to rotate about the blade rotation pin 330. The blade slot 340 may be curved to along the blade to rotate upon actuation of the cutting apparatus actuation mechanism 320.

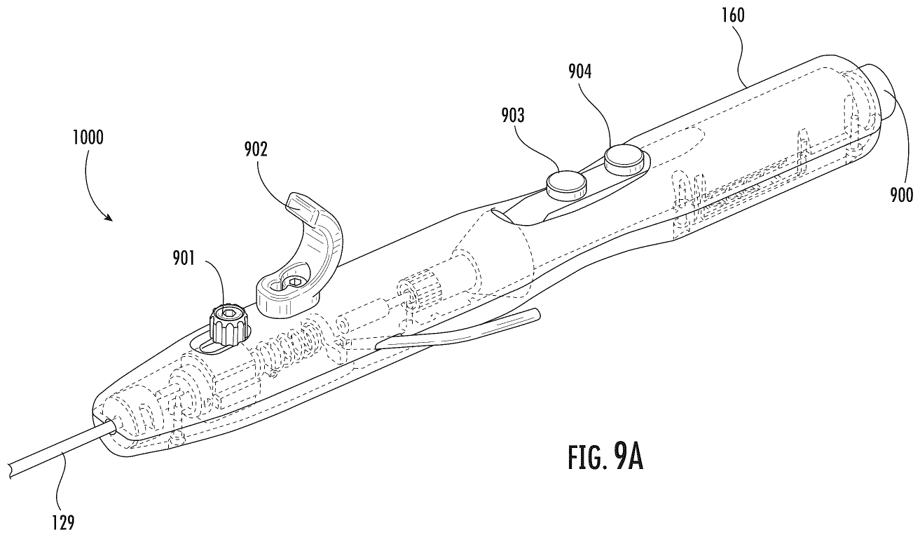

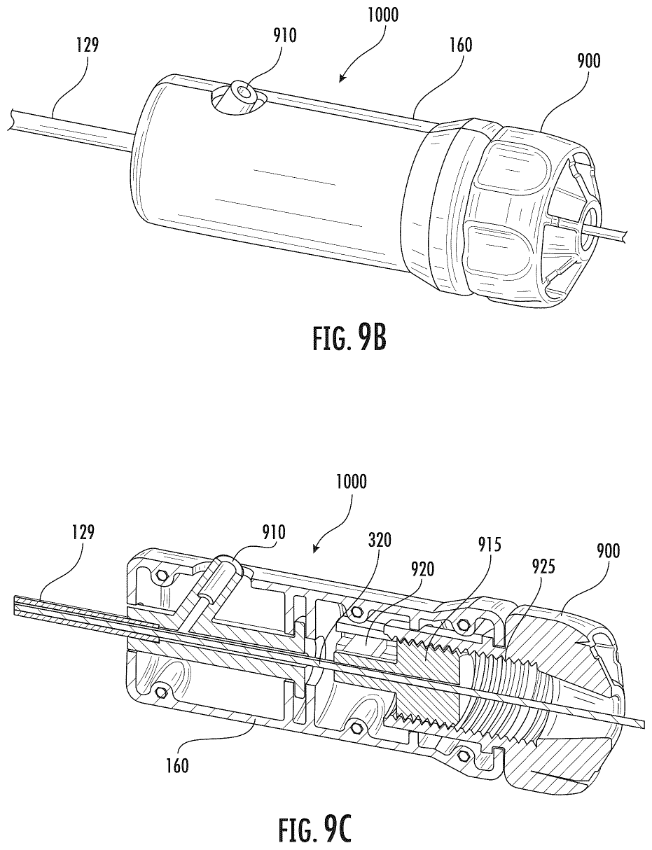

The cutting apparatus actuation mechanism 320, as shown, may be a rod or connector that passes through a through-hole in the translation body 315 and is moveably attached to the blade slot 340. As the cutting apparatus actuation mechanism 320 is moved in the direction of the blade 305, the blade 305 is caused to rotate in the clockwise direction, resulting in the blade 305 moving to the deployed position (e.g., protruding from the incision channel). As the cutting apparatus actuation mechanism 320 is moved in the direction opposite of the blade 305, the blade 305 is caused to rotate in the counter-clockwise direction, resulting in the blade 305 moving to the retracted position (e.g., disposed within the incision channel). The cutting apparatus actuation mechanism 320 may be controlled via a multi-lumen catheter controller, such as the controller shown in FIGS. 9A and 9B.

Upon movement of the blade from the retracted position to the deployed position, the retractable cutting apparatus 300 can be moved via the retractable cutting apparatus movement actuator 325. The retractable cutting apparatus movement actuator 325 is fixed at one end to the translation body 315. The retractable cutting apparatus movement actuator 325 may be controlled via a multi-lumen catheter controller, such as the controller shown in FIGS. 9A and 9B.

The blade 305 may be curved to allow the blade to puncture pericardium tissue (e.g., puncture into the parietal layer 55) when being rotated into the deployed position (e.g., the blade is curved in a direction of rotation from the retracted position to the deployed position). The blade is structured to have a sufficient force to puncture pericardial tissue in an instance in which the blade is moved from the retracted position to the deployed position. The blade 305 may have a cutting surface 310 that is positioned to cut tissue upon being moved within the human body in an instance in which the blade 305 is in the deployed position. The cutting surface 310 of the blade 305 when laterally projected (e.g., in the deployed position), faces away from the distal end and towards the proximal end of the multi-lumen catheter.

One or more components of the medical device may be radiopaque to allow the medical device to be seen within the human body using ultrasound. In various embodiments, portions of the retractable cutting apparatus, multi-lumen catheter, and/or introducer may be radiopaque.

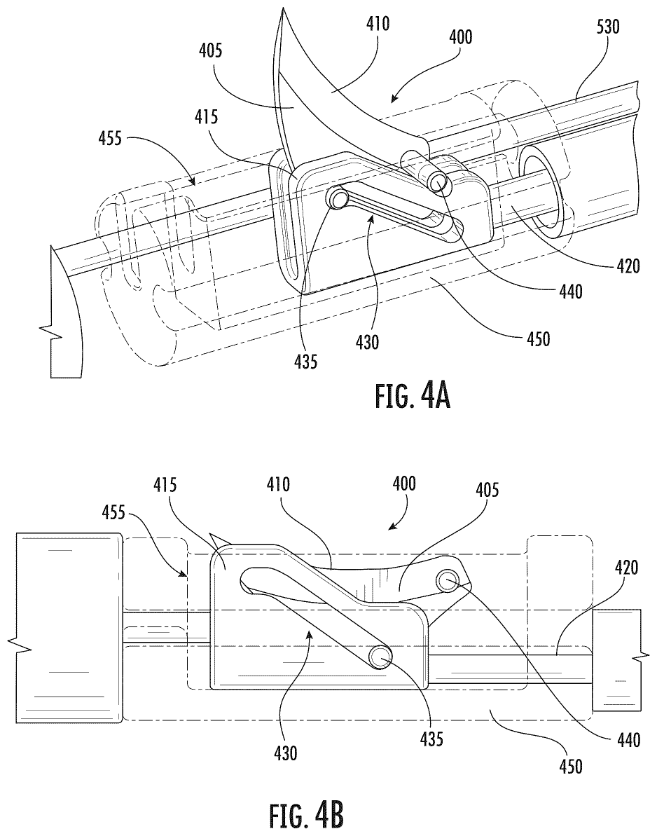

FIGS. 4A-6B illustrate another example retractable cutting apparatus 400 to be used in a medical device discussed herein. FIG. 4A shows a transparent view of the retractable cutting apparatus 400 in the deployed position. FIG. 4B shows a transparent view of the retractable cutting apparatus 400 in the retracted position.

As shown in FIGS. 4A and 4B, the retractable cutting apparatus 400 includes a cutting apparatus casing 450, a translation body 415, a blade 405, and a retractable cutting apparatus movement actuator 420. The cutting apparatus casing 450 defines a translation body channel 455 that is structured to receive the translation body 415. As such, the translation body 415 moves within the translation body channel 455. The cutting apparatus casing 450 can then move within the incision channel (shown in FIG. 2).

The translation body 415 defines a translation body slot 430 that is structured to receive a blade movement pin 435. The blade movement pin 435 is attached to the blade 405, such that the blade moves based on the position of the blade movement pin 435 within the translation body slot 430. The translation body slot 430 may be slanted to allow the blade movement pin 435 (and the blade 405) to move in the latitudinal direction.

The blade 405 is rotatably attached to the cutting apparatus casing 450. As shown in FIGS. 4A and 4B, the blade 405 may be rotatably attached to the cutting apparatus casing 450 via a blade rotation pin 440. The blade rotation pin 440 may be received by an aperture in the cutting apparatus casing 450. The blade 405 rotatably fixed about the blade rotation pin 440 and moves based on the movement of the translation body 415.

The retractable cutting apparatus movement actuator 420 is attached to the translation body 415. Upon movement of the retractable cutting apparatus movement actuator 420 in the direction of the proximal end of the multi-lumen catheter, the blade movement pin 435 attached to the blade 405 moves in the opposite direction along the translation body slot 430 causing the blade 405 to rotate in the clockwise direction (about the blade rotation pin 440). As such, the blade 405 moves from the retracted position to the deployed position (e.g., the blade 405 rotates in the clockwise direction until the blade 405 is deployed). The retractable cutting apparatus movement actuator may be actuated by a multi-lumen catheter controller, such as the one shown in FIGS. 9A-9C

To move the blade 405 from the deployed position to the retracted position, the retractable cutting apparatus movement actuator 420 is moved in the opposite direction of the proximal end of the multi-lumen catheter (e.g., in the direction of the introducer 115). As the retractable cutting apparatus movement actuator 420 is moved in the opposite direction of the proximal end of the multi-lumen catheter, the blade movement pin 435 attached to the blade 405 moves in the opposite direction along the translation body slot 430 (e.g., towards the proximal end of the multi-lumen catheter) causing the blade 405 to rotate in the counterclockwise direction (about the blade rotation pin 440). As such, the blade 405 moves from the deployed position to the retracted position (e.g., the blade 405 rotates in the counterclockwise direction until the blade 405 is retracted).

In various embodiments, the retractable cutting apparatus may have one or more predefined positions between the fully retracted position and the fully deployed position. For example, the blade 405 may have a range of rotation about the blade rotation pin 440. In the retracted position (shown in FIG. 4B), the blade 405 may be approximately parallel with the longitudinal axis (e.g., along the multi-lumen catheter). The blade 405 can rotate in the clockwise direction for the range of rotation. In some embodiments, the range of rotation of the blade 405 may be from approximately 60 degrees to approximately 120 degrees. In some embodiments, the range of rotation of the blade 405 may be from approximately 70 degrees to approximately 110 degrees. In some embodiments, the range of rotation of the blade 405 may be from approximately 80 degrees to approximately 110 degrees. In some embodiments, the range of rotation of the blade 405 may be from approximately 80 degrees to approximately 100 degrees. In some embodiments, the range of rotation of the blade 405 may be approximately 90 degrees

The blade 405 may be curved to allow the blade to puncture pericardium tissue (e.g., puncture into the parietal layer 55) when being rotated into the deployed position (e.g., the blade is curved in a direction of rotation from the retracted position to the deployed position). The blade is structured to have a sufficient force to puncture pericardial tissue in an instance in which the blade is moved from the retracted position to the deployed position. The blade 405 may have a cutting surface 410 that is positioned to cut tissue upon being moved within the human body in an instance in which the blade 305 is in the deployed position. The cutting surface 310 of the blade 305 when laterally projected (e.g., in the deployed position), faces away from the distal end and towards the proximal end of the multi-lumen catheter.

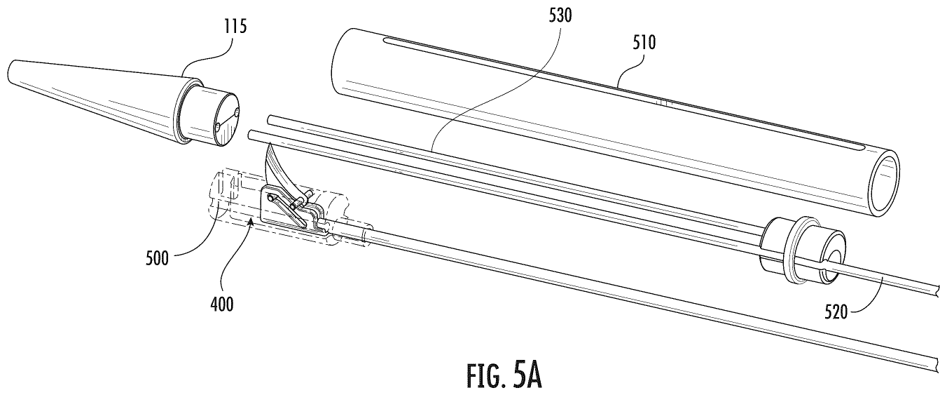

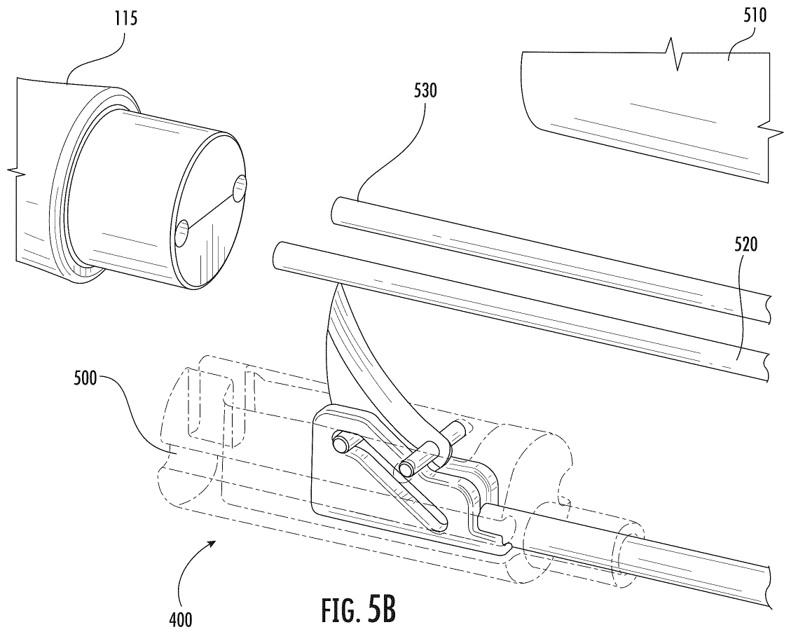

FIGS. 5A and 5B are exploded views of a medical device with the retractable cutting apparatus 400 shown in FIGS. 4A and 4B. The cutting apparatus casing defines one or more guidewire channels 500 that are defined to configured to receive a guidewire. The retractable cutting apparatus 400 follows travels along the one or more guidewires 520, 530 within the incision channel. The incision device may also have a cover tubing 510 that defines the incision channel therein.

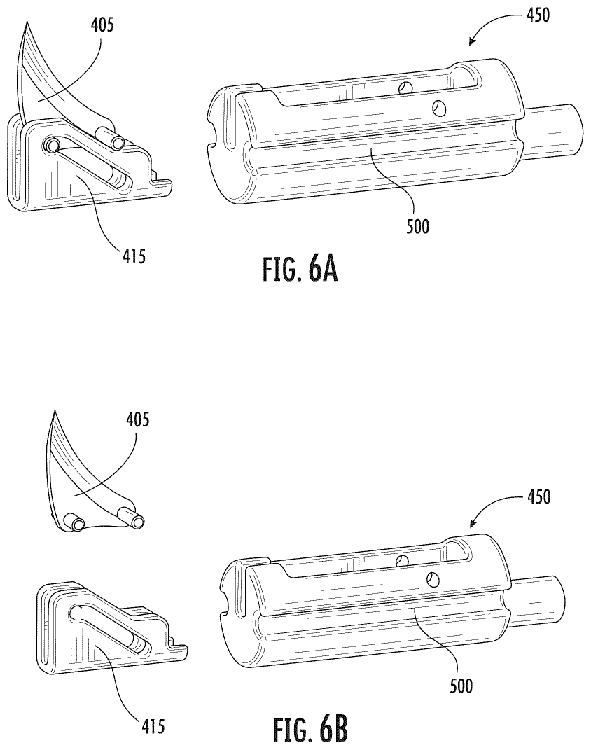

FIGS. 6A and 6B is an exploded view of the retractable cutting apparatus 400. As shown, the blade 405, the translation body 415, and the cutting apparatus casing 450 may each be an individual component that is assembled to form the retractable cutting apparatus 400. The blade 405 is moveably coupled to the translation body 415 and the translation body 415 is slidably disposed within the cutting apparatus casing 450.

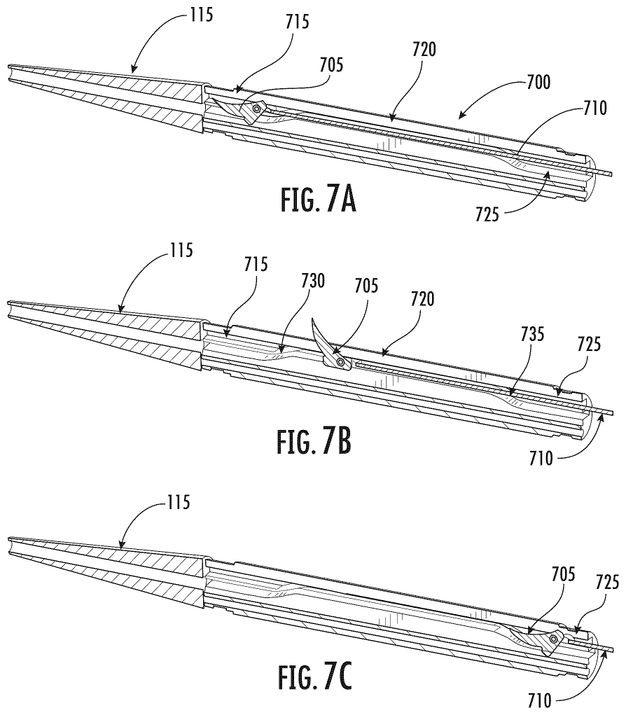

FIGS. 7A-7C illustrate various position of a blade within another example retractable cutting apparatus 700. As shown, the incision channel is varying shapes to cause the blade to deploy or retract at given locations along the incision channel. The retractable cutting apparatus 700 includes a blade 705 attached to a cutting apparatus actuation mechanism 710 (e.g., a wire or other connecter). The cutting apparatus actuation mechanism 710 moves the blade 705 along the incision channel causing the blade to move from the retracted position to the deployed position and then back to the retracted position.

The incision channel defines a first retracted area 715 that defines a depth that is sufficient for the blade 705 to be disposed therein. As show in FIG. 7A, in an instance in which the blade 705 is within the first retracted area, the blade is in the retracted position (e.g., disposed within the incision channel. As the cutting apparatus actuation mechanism 710 moves the blade in the lateral direction, the blade engages the deployment ramp 730 that is defined between the first retracted area 715 and the deployed area 720. Upon engagement with the deployment ramp 730, the blade 705 is caused to rotate in the clockwise direction. As the depth of the deployed area 720 is less than the height of the blade 705, the blade 705 is caused to remain in the deployed position while traveling through the deployed area 720, as shown in FIG. 7B. Upon reaching the retraction ramp 735 defined between the deployed area 720 and the second retracted area 725, the blade 705 then rotates in the counterclockwise direction into the retracted position in the second retracted area 725. As such, the blade 705 begins in the retracted position in the first retracted position (e.g., during placement of the incision device within the pericardium), then deploys and cuts the parietal layer of the pericardium while in the deployed area 720 before returning to the retracted position in the second retracted area 725 (e.g., to allow for the incision device to be removed). The depth of the first retracted area 715 and/or the second retracted area 725 may be based on the size of the blade 705. For example, the depth of the first retracted area 715 and/or the second retracted area 725 may be at least the same as the width of the blade 705 to allow for the blade 705 to be disposed within the given retracted area.

The blade 705 may be curved to allow the blade to puncture pericardium tissue (e.g., puncture into the parietal layer 55) when being rotated into the deployed position (e.g., the blade is curved in a direction of rotation from the retracted position to the deployed position). The blade is structured to have a sufficient force to puncture pericardial tissue in an instance in which the blade is moved from the retracted position to the deployed position. The blade 705 may have a cutting surface that is positioned to cut tissue upon being moved within the human body in an instance in which the blade 705 is in the deployed position. The cutting surface of the blade 705 when laterally projected (e.g., in the deployed position), faces away from the distal end and towards the proximal end of the multi-lumen catheter.

The blade 705 may also have a counterweight (not shown) defined or attached to the bottom of the blade that assists in moving the blade between the retracted position and the deployed position. For example, the counterweight may cause the blade 705 to rotate into the retracted position in the second retracted area 725 and the counterweight may also assist the blade to remain upright in the deployed area 720 (e.g., during cutting by the blade).

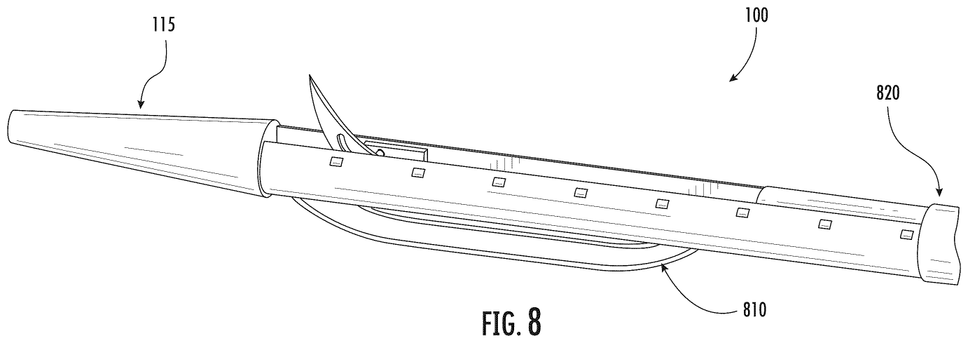

FIG. 8 illustrates a medical device of various embodiments with stabilizing members 810. Various embodiments of the medical devices discussed herein may have stabilizing members 810. The stabilizing member 810 may be independently user controlled by advancing actuating wire distally toward introducer 115 tip that laterally extends member 810 a distance from the multi-lumen catheter. In one example, two or more stabilizing members 810 are positioned radially about the assembly. In one example, two or more stabilizing members 810 are positioned radially about the assembly about 120 degrees apart. In one example, the two or more stabilizing members 810 are offset longitudinally from the cutting surface to minimize or eliminate pushing the device through the newly cut slit in the parietal layer 55 of the pericardium 60 just as it is formed. Stabilizing members 810 can be flexible rods or strip, or inflatable structures, such as balloons that can be inflated with air or liquid (saline).

The one or more stabilizing members 810 are configured to move between a stabilizing retracted position and a stabilizing deployed position upon positioning of the multi-lumen catheter within the human body.

The medical device 100 may also include a sheath 820 configured to moveably cover the incision channel. The sheath is configured to cover the incision channel in an instance in which the multi-lumen catheter is not positioned within human body. Once the multi-lumen catheter has been positioned (e.g., the incision device is positioned where the cut in the parietal layer 55 of the pericardium 60 is to be made), the sheath may be actuated to uncover the incision channel. In some embodiments, the sheath 820 may not be moveable and merely has a slit to allow the knife to extrude. The sheath 820 can also hold the one or more stabilizing members in the stabilizing retracted position during movement (e.g., the sheath moves to uncover the stabilizing member(s) upon placement in the pericardium.

With reference to FIGS. 9A, the controller 1000 is shown as a handle 160. The controller may have multiple actuating knobs 900, 901, 902 and actuating buttons 903, 904 for controlling the multi-lumen catheter 129 and the various components of the medical device 100 (e.g., actuating the sheath 820, actuating stabilizing members 810). The actuating knob 900 may correspond to the actuating knob 900 discussed below in reference to FIGS. 9B and 9C. Knob 901 may be configured to rotate to extend or retract the catheter 129. Knob 902 may be engaged to activate one or more components on the medical device (e.g., actuating the stabilizing member(s)). Similarly, the actuating buttons 903, 904 may be engaged to activate various components of the multi-lumen catheter (e.g., the device may use RF electrode cutting as well and the actuating buttons 903, 904 may be used. Various other controllers may be contemplated that allow for the retractable cutting apparatus to be deployed and retracted.

A method of manipulating medical device 100 would include providing medical device 100 engaged with controller 1000 and controlling, for example, manipulating any one or combination of multiple actuating knobs 900, 901, 902 and actuating buttons 903, 904 to provide at least one of at least one of movement of the blade between the retracted position and the deployed position, movement of the retractable cutting apparatus along the incision channel, movement of the catheter, movement of stabilizing member(s), energy to the incision device, and/or the like with controller 1000. The aforementioned method is applicable to any previously disclosed transection devices.

In one example, the controller 1000 allows the operation of various potential operations of the incision device 140, including biasing/clamping the distal blade open and closed, which can be achieved by an appropriate mechanism configured to pull/push a rod. In one example, there is a mechanism used to release/retrieve balloons/nitinol components that function to stabilize and apply counterpressure for the incision device 140 and its components, including the cutting surface. Controller 1000 may comprise one or buttons used to operate and control the electrosurgical features of the device.

As shown in FIGS. 9B-C, the controller 1000 may include a handle 160 with an actuating knob 900. The controller 1000 may be integrated with the example embodiment discussed above in reference to FIG. 9A, such that the handle 160 includes one or more buttons or controls discussed above. The controller 1000 shown in FIG. 9B may have a handle 160 with an actuating knob 900. The controller 1000 also includes a liquid inlet 910 that is configured to allow a liquid, such as a contrast substance, to be pass through the multi-lumen catheter 129 and into the human body. The controller 1000 is connected to the multi-lumen catheter 129 to operate the multi-lumen catheter 129 and/or the incision device 140.

As shown in FIG. 9C, the actuating knob 900 is rotatably attached to the handle 160, such that the actuating knob 900 is rotated relative to the handle 160. The actuating knob 900 includes an internal threading 925 that is configured to receive an internal screw 915 that is configured to move based on the rotation of the actuating knob 900. The internal screw 915 is attached to the cutting apparatus actuation mechanism 320, such that the movement of the internal screw 915 causes the cutting apparatus actuation mechanism 320 to also move, which subsequently moves the retractable cutting apparatus between the retracted position and the deployed position (e.g., the blade 305 is moved to be exposed outside of the catheter). While FIG. 9C illustrates the cutting apparatus actuation mechanism 320 shown in FIGS. 3A-3D, the same controller design could be used for the retractable cutting apparatus movement actuator 420 shown in FIGS. 4A-5B and/or the cutting apparatus actuation mechanism 710 shown in FIGS. 7A-7C.

As the actuating knob 900 is turned, the threaded screw 915 moves along the longitudinal axis (e.g., along the internal threading 925). In an instance in which the actuating knob 900 is moved in the clockwise direction, the threaded screw 915 moves in the direction opposite of the multi-lumen catheter 129, causing the cutting apparatus actuation mechanism 320 to also move in the direction of the threaded screw 915 (e.g., in the direction opposite of the distal end of the multi-lumen catheter 129). Referring to the embodiment shown in FIGS. 3A-3D, the movement of the cutting apparatus actuation mechanism 320 in the direction opposite the multi-lumen catheter causes the retractable cutting apparatus (e.g., the retractable cutting apparatus 300) to be moved from the retracted position to the deployed position. Alternatively, in the embodiment shown in FIGS. 4A-5B, the movement of the retractable cutting apparatus movement actuator 420 in the direction opposite the multi-lumen catheter causes the retractable cutting apparatus (e.g., the retractable cutting apparatus 400) to be moved from the deployed position to the retracted position. Referring to the embodiment shown in FIGS. 7A-7C, the movement of the cutting apparatus actuation mechanism 710 in the direction opposite the distal end of the multi-lumen catheter causes the blade 705 to move in the same direction (e.g., moving from the retracted position to the deployed position and then back to the retracted position).

In an instance in which the actuating knob 900 is rotated in the counterclockwise direction, the threaded screw 915 moves in the direction of the multi-lumen catheter 129 (e.g., in the direction of the distal end of the multi-lumen catheter 129) causing the cutting apparatus actuation mechanism 320 to move in the direction of the distal end of the multi-lumen catheter 129. Referring to the embodiment shown in FIGS. 3A-3D, the movement of the cutting apparatus actuation mechanism 320 in the direction of the multi-lumen catheter causes the retractable cutting apparatus (e.g., the retractable cutting apparatus 300) to be moved from the deployed position to the retracted position. Alternatively, in the embodiment shown in FIGS. 4A-5B, the movement of the retractable cutting apparatus movement actuator 420 in the direction opposite the multi-lumen catheter causes the retractable cutting apparatus (e.g., the retractable cutting apparatus 400) to be moved from the retracted position to the deployed position. Referring to the embodiment shown in FIGS. 7A-7C, the movement of the cutting apparatus actuation mechanism 710 in the direction of the distal end of the multi-lumen catheter causes the blade 705 to move in the same direction.

The threaded screw 915 has a screw stopper 920 that stops the movement of the threaded screw 915 in the direction opposite the multi-lumen catheter 129. As such, the screw stopper 920 is positioned to hit an internal protrusion of the handle 160 once the retractable cutting apparatus is moved into the deployed position. In various embodiments, the range of movement of the threaded screw 915 is determined by the screw stopper 920, such that the threaded screw 915 does not travel along the entire internal threading 925. In various embodiments, the screw stopper 920 may be adjustable to change the angle or position of the blade upon deployment.

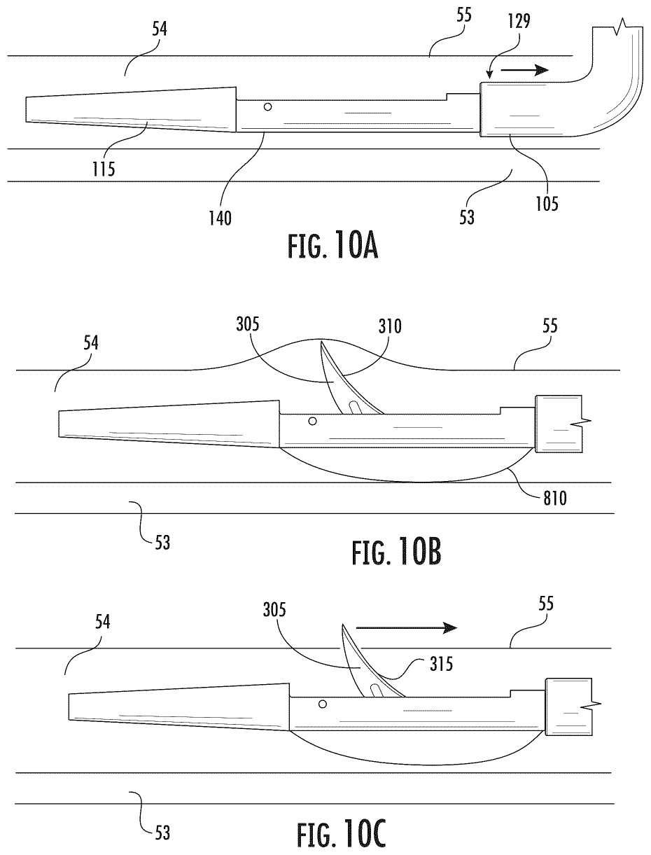

FIGS. 10A-10C illustrate medical device 100 being deployed. In one example, device 100 is deployed subxiphiodally, e.g., through percutaneous access achieved by directing a needle into the patient under the xiphoid process towards the pericardial cavity. In another example, device 100 is deployed intravascularly thru the right ventricle. The medical device 100 can be advanced through a subject's pericardial cavity 54 in undeployed configuration with the blade 305 in the retracted position within the incision channel. While the blade 305 is shown in FIGS. 10A-10C, each of the blades (e.g., blade 405 and blade 705) disclosed herein may be operated to cause the incision using operations discussed in reference to FIGS. 10A-10C (e.g., once the blades are moved to the deployed position, the parietal layer 55 of the pericardium 60 is punctured, and then the blade moves along the parietal layer 55 of the pericardium 60 while the multi-lumen catheter remains stationary within the pericardial cavity 54).

The initial access to the pericardial cavity 54 may be achieved via the introducer 115. As the medical device is positioned within the pericardial cavity 54, the sheath 820 may be covering the incision channel. As the multi-lumen catheter is positioned within the pericardial cavity 54 at the location of the incision, the sheath 105 may be removed, as shown in FIG. 9A. The sheath may uncover the incision device 140 that is still in the retracted position.

As shown in FIG. 9B, the medical device 100 may have one or more stabilizing members 810. The stabilizing member 810 may be independently user controlled by advancing an actuating wire distally toward introducer 115 tip that laterally extends stabilizing member(s) 810 a distance from medical device 100. In one example, two or more stabilizing members 810 are positioned radially about the assembly. In one example, two or more stabilizing members 810 are positioned radially about the assembly about 120 degrees apart. In one example, the two or more stabilizing members 810 are offset longitudinally from the cutting surface to minimize or eliminate pushing the device through the newly cut slit in the parietal layer 55 of the pericardium 60 just as it is formed. Stabilizing members 810 can be flexible rods or strip, or inflatable structures, such as balloons that can be inflated with air or liquid (saline).

The one or more stabilizing members 810 are configured to move between a stabilizing retracted position and a stabilizing deployed position upon positioning of the multi-lumen catheter within the human body.

The retractable cutting apparatus 300 may be moved from the retracted position to the deployed position as discussed herein. The retractable cutting apparatus 300 uses the puncture tip of the blade 305 to puncture the pericardial tissue to be cut. After the blade 305 punctures the pericardial tissue, the incision can be made along the pericardium tissue (e.g., the parietal layer 55). The incision is made by moving the retractable cutting apparatus along the incision channel. The multi-lumen catheter 129 remains stationary within the pericardial cavity 54 during the incision by the blade. As such, the incision length is defined as the distance in which the retractable cutting apparatus moves along the incision channel.

The tip of blade 305 can serve as a radiopaque marker indicating ‘bunching up’ of tissue, for example, due to excessive pull before a through-cut is achieved. In one example, the back silhouette of the blade may be selectively insulated (as it may be activated to achieve the exit puncture) to avoid ablation damage to neighboring anatomy.

Upon puncture of the parietal layer 55 by the blade, the retractable cutting apparatus is moved along the pericardial cavity 54 to cause the incision for the incision length intended. The incision is a linear incision along the parietal layer 55. Upon moving the blade 305 the length of the intended incision, the retractable cutting apparatus is moved back to the retracted position and the sheath 105 can be moved to cover the incision channel. The stabilizing member(s) 810 may also be moved to the stabilizing retracted position. The multi-lumen catheter can then be removed from the human body without having any blades exposed.

In one example, a puncture to deliver a guidewire into the pericardial space is performed through heart tissue. When a transvascular approach through the RAA, IVC, or SVC is employed, a closure device may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

The following exemplary occlusion descriptions relate to a transvascular approach through the RAA, IVC, or SVC using one of the aforementioned transection devices. In one example, an introducer/dilator 115 delivers a wire into the pericardial space through heart tissue. A closure or occlusion device is introduced for hemostasis during the procedure. The closure or occlusion device in one example includes outward or radially directed splines deployed in an expanded configuration. When the guide catheter is removed, the splines or radial members of the closure device contract inwardly towards the unstressed state of the transection device in order to close and seal the opening. The closure device is designed such that a pericardial cutting device can pass through and into the pericardial space.

In one example, at least one incision length is made in the pericardium of a heart. The at least one incision lengths, in a heart with a dysfunction treatable with the present method, may cause the pericardium to separate radially about the cut line, without the need for removal of pericardial tissue. Combinations of incision lengths and paths, and combination of incision lengths and paths with one or more of partial removal of pericardium, drainage, and other pericardial treatments can be employed.

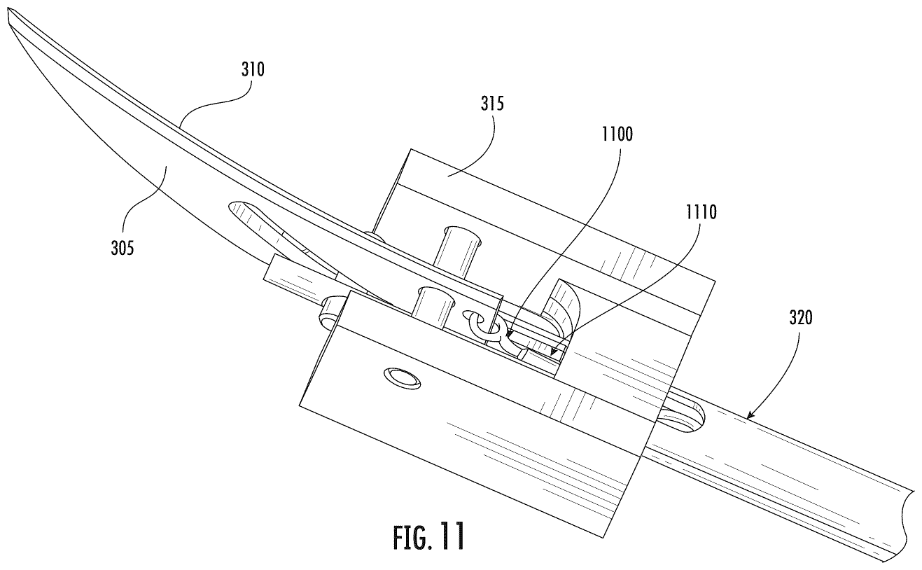

Referring now to FIG. 11, an example retractable cutting apparatus is shown with RF capabilities. In various embodiments, the blade (e.g., blade 305) may be provided an electrical current that disrupts the human tissue. The electrical current may be used in addition to a sharp edge (e.g., cutting surface 310). Alternatively, the electrical current may be used in place of the sharp edge. For example, the blade 305 may not have a sharp edge. The electrical current may be supplied to the blade 305 via an electrical transmitting source 1100 (e.g., a copper wire). The electrical transmitting source 1100 may have an insulation area (e.g., an insulation layer 1110) that insulates the rest of the medical device 100 from the electrical current (e.g., only the blade 305 receives the electrical current. For example, the translation body 315 and the rest of the multi-lumen catheter 129 may be isolated from the electrical transmitting source 1100 (e.g., the exposed copper wire) and therefore non-electrified. While FIG. 11 shows the embodiment shown in FIGS. 4A-4D, the other retractable cutting apparatuses 400, 700 may also be electrified using the same or similar electrical transmitting source 1100 (e.g., a copper wire may be attached to the blade 405 or the blade 705).

In one example, creating at least one incision length is determined in response to a signal indicative of a reduction of restraint of the heart. In one example, creating at least one incision length is determined in response to a signal indicative of a reduction of restraint of the heart; and repeating the creating of at least one incision length. In one example, the presently disclosed method further comprises, after creating the at least one incision length, confirming a location of a distal end of the multi-lumen catheter device; and in response to a signal indicative of a reduction of restraint of the heart, repeating the steps of creating the at least one incision length, and confirming a location of the distal end.

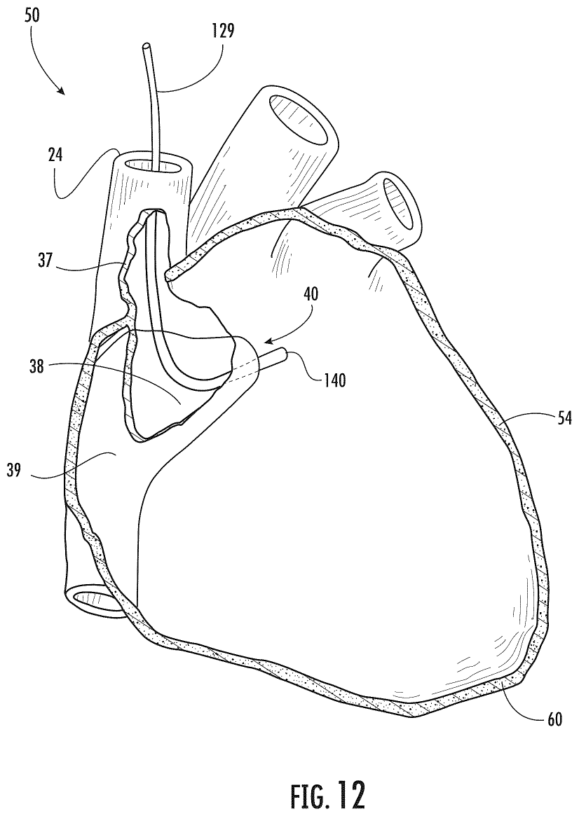

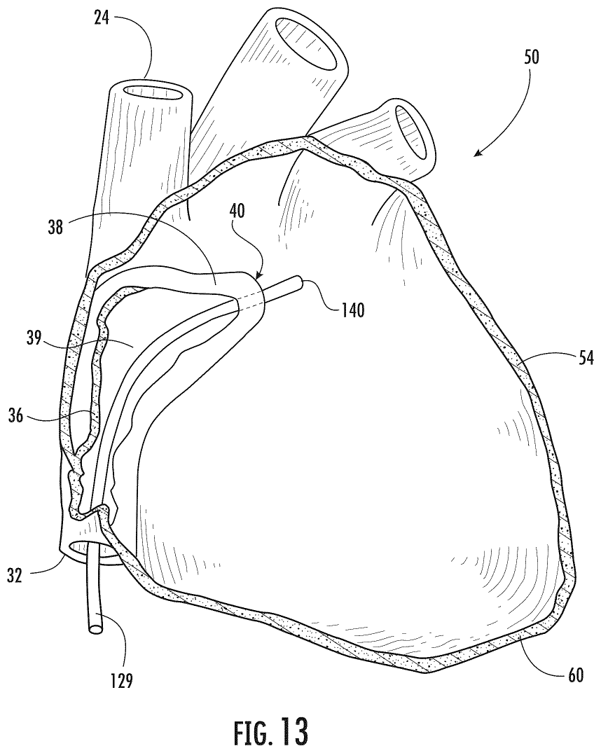

FIGS. 12 and 13 shows exemplary intravascular approaches for delivering the transection devices of the present disclosure to the pericardial cavity 54. Thus, FIG. 12 depicts heart 50 viewed in isolation from the body, with the pericardium 60 or pericardial sac encasing the cardiac muscle (i.e., epicardium, myocardium and endocardium). The small space which is present between the heart muscle and pericardium 60 represents the pericardial cavity 54.

The presently disclosed transection devices that can be presented to the pericardial cavity 54. In one example via the right atrial appendage 38 (RAA), which is a suitable site for entry into the pericardial cavity 54, is used. Right atrial appendage 38 lies tangential to and between pericardium 60 and the epicardium/epicardial adipose tissue 57. In one example, any of the presently disclosed devices is guided into right atrial appendage 38 via right atrium 39 so as to be positioned substantially in parallel with the wall of pericardium 60 such that when the wall of right atrial appendage 38 is pierced by any of the presently disclosed devices substantially without risk of damaging the epicardium or other heart tissue. Other access routes to the pericardial cavity can be used, for example, direct “puncture out” of SVC or IVC/coronary sinus (CS) and a “puncture into” the pericardium.

In some examples, right atrial appendage 38 is accessed via conventional vena cava routes. FIG. 12 illustrates entry of any of the presently disclosed devices into right atrium 39 via the superior vena cava 24 (SVC). A cut-away 37 shows passage of any of the presently disclosed devices through superior vena cava 24, right atrium 39, and right atrial appendage 38. A distal tip of catheter 129 is shown exiting right atrium 39 at apex 40.

FIG. 8 illustrates an alternative entry of any of the previously disclosed devices into right atrium 39 via the inferior vena cava 32 (IVC). A cut-away 36 shows passage of catheter 129 through inferior vena cava 32, right atrium 39, and right atrial appendage 38. A distal tip of catheter 129 is shown exiting right atrium 39 at apex 40.

Thus, by way of example, a method of reducing pericardial restraint of a subject in need thereof using any of the presently disclosed devices is provided by the following steps. Any of the presently disclosed devices is maneuvered through one of the vena cava 24, 32 to right atrium 39. Once inside right atrium 39, any of the presently disclosed devices is passed into the right atrial appendage 38. The wall of right atrial appendage 38 is pierced at apex 40, and the catheter is advanced into the pericardial cavity 54. Other transvascular-right heart routes to the pericardial cavity 54 are envisaged. Furthermore, left atrial appendage, coronary sinus, and right ventricle pathways are envisaged for transvascular access to the pericardial cavity 54.

Note that the wall of the right atrial appendage may be pierced with any of the presently disclosed devices itself, or with an instrument (e.g., guidewire) passed through a lumen of the any of the presently disclosed devices, e.g., over the wire. Further, any of the previously disclosed devices may be passed into the pericardial space through the opening in the wall of the atrial appendage, or an instrument passed through the lumen of any of the presently disclosed devices may be presented into the pericardial cavity 54. These details will depend on the procedure being performed and on the type of the previously disclosed device being employed.

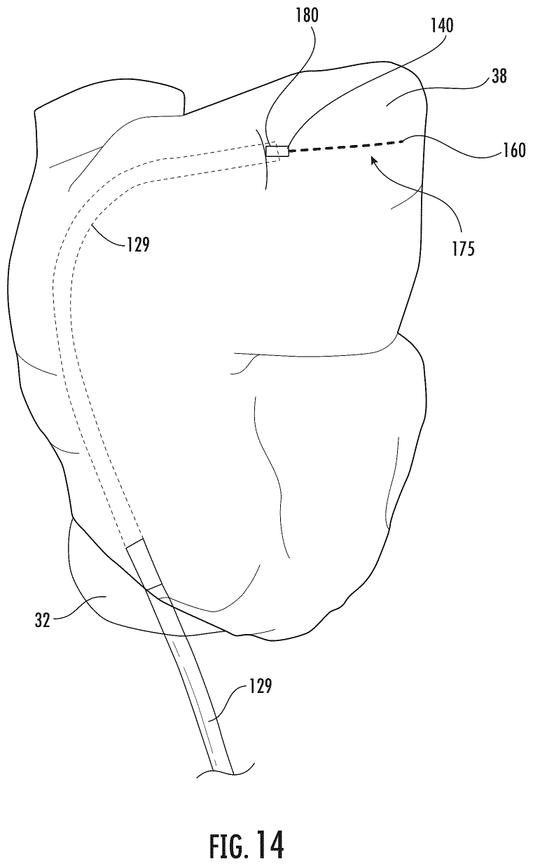

As shown in FIG. 14, any of the presently disclosed devices can be used to create a cut path of a length in a pericardium, e.g., in a parietal layer 58. Thus, a catheter 129, e.g., a steerable catheter can be employed, extending through the IVC, through the RA, and into the RAA, for example, and then into the pericardial cavity 54. The catheter 129 may have one or more steerable segments guiding any of the presently disclosed devices, with a radius of curvature of between about 1 inch and about 5 inches, with an arc length of between about 90° and about 180°. As exemplary shown in FIG. 21, any of the presently disclosed devices can be positioned in the pericardial cavity 54 and can begin a cut path 175 at a start point 165 and ends at endpoint 180 of a length. At least a portion of the parietal layer 58 of the pericardium, and the fibrous pericardium 56, and pericardial adipose tissue 59 are separated along cut path 175. The one or more incisions along the lengths, in a heart with a dysfunction treatable with the present method, cause the pericardium to separate radially about the cut line of the incision path 175, without the removal of pericardial tissue and with a reduction in pericardial restraint. One or more cut paths 175 can be made, and different cut paths, of various lengths can be used to reduce pericardial restraint. In one example, the cut path 175 and its length is pre-operatively determined. Other cut paths and lengths can be used.

In one example, the presently disclosed device further comprises at least one nerve detection device. In one example, the at least one nerve detection device is located on the catheter 129. In one example, the at least one nerve detection device is located adjacent the incision device. In one example, the at least one nerve detection device is located on the dilator/introducer 115. In one example, the at least one nerve detection device is located on the cutting surface.

Any one of the presently disclosed devices can further comprises at least one nerve stimulation device. In one example, the at least one nerve stimulation device is located on the catheter 129. In one example, the at least one nerve stimulation device is located adjacent the incision device. In one example, the at least one nerve stimulation device is located on dilator/introducer 115. In one example, the at least one nerve stimulation device is located on the cutting surface.

In one example, the presently disclosed devices discussed above further comprise an optical channel in the multi-lumen catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen catheter to accommodate a lens coupled to a fiber optic cable, optionally with a light source, e.g., an LED.

A kit, comprising any one of the presently disclosed medical devices, a sheath, a guidewire, and an introducer/dilator is provided.

While certain embodiments of the present disclosure have been illustrated with reference to specific combinations of elements, various other combinations may also be provided without departing from the teachings of the present disclosure. Thus, the present disclosure should not be construed as being limited to the particular exemplary embodiments described herein and illustrated in the Figures, but may also encompass combinations of elements of the various illustrated embodiments and aspects thereof.

We claim:

1. A medical device for creating elongated incisions within a pericardium, the device comprising:
an incision device operably coupled to a distal end of an elongate catheter, wherein the incision device comprises an incision channel defined along a lateral side of the elongate catheter; and
a retractable cutting apparatus disposed within the incision channel, wherein the retractable cutting apparatus comprises a blade structured to move between a retracted position and a deployed position,
wherein upon movement of the blade into the deployed position, the retractable cutting apparatus is structured to move along the incision channel while the incision device remains stationary.

2. The medical device of claim 1, wherein the retractable cutting apparatus comprises:
a translation body operably coupled to the blade;
a blade rotation pin rotatably connecting the blade to the translation body, wherein the blade rotates about the blade rotation pin; and
an actuation pin attached to a cutting apparatus actuation mechanism structured to move the blade between the retracted position and the deployed position, wherein the actuation pin moves along a blade slot defined on the blade, wherein the movement of the actuation pin along the blade slot causes the blade to move between the retracted position and the deployed position,
wherein in an instance in which the blade is in the retracted position, the blade is disposed within the incision channel, and
wherein in an instance in which the blade is in the deployed position, the blade at least partially protrudes from the incision channel.

3. The medical device of claim 2, wherein the blade slot is curved.

4. The medical device of claim 1, wherein the blade of the cutting apparatus is curved in a direction of rotation from the retracted position to the deployed position.

5. The medical device of claim 1, further comprising:
a translation body operably coupled to the blade; and
a retractable cutting apparatus movement actuator attached to the translation body, wherein the retractable cutting apparatus movement actuator is movable to move the retractable cutting apparatus along the incision channel.

6. The medical device of claim 2, further comprising a retractable cutting apparatus movement actuator attached to the translation body, wherein the retractable cutting apparatus movement actuator is movable to move the retractable cutting apparatus along the incision channel.

7. The medical device of claim 1, further comprising an introducer positioned near a distal end of the incision device.

8. The medical device of claim 7, wherein at least a portion of the introducer is radiopaque.

9. The medical device of claim 1, wherein a cutting surface of the blade when laterally projected, faces away from the distal end and towards a proximal end of the catheter.

10. The medical device of claim 1, further comprising one or more stabilizing members structured to maintain the catheter at a given location.

11. The medical device of claim 10, wherein the one or more stabilizing members are structured to move between a stabilizing retracted position and a stabilizing deployed position.

12. The medical device of claim 1, wherein at least a portion of a cutting surface of the blade is reversibly adjustable laterally relative to a longitudinal axis of the catheter between a range of angles.

13. The medical device of claim 1, wherein a plurality of radiopaque markers is provided along an edge of the incision channel, wherein a position of the blade can be determined based on the radiopaque markers.

14. The medical device of claim 1, wherein the retractable cutting apparatus comprises:
a translation body operably coupled to the blade, wherein the translation body defines a translation body slot;
a blade movement pin attached to the blade, wherein the blade movement pin is positioned to move within the translation body slot, wherein at a first position within the translation body slot, the blade is in the retracted position and at a second position within the translation body slot, the blade is in the deployed position; and
a cutting apparatus casing defining a translation body channel that allows the translation body to move within the translation body channel;
wherein the blade is rotatably attached to the cutting apparatus casing, wherein the blade is structured to rotate based on the movement of the translation body,
wherein upon moving the translation body in a first direction, the blade movement pin moves along the translation body slot causing the blade to rotate from a clockwise direction in the retracted position to the deployed position,
wherein in an instance in which the blade is in the retracted position, the blade is disposed within the incision channel, and
wherein in an instance in which the blade is in the deployed position, the blade at least partially protrudes from the incision channel.

15. The medical device of claim 1, further comprising a controller engaged to the incision device, wherein the controller provides at least one of: (i) movement of the blade between the retracted position and the deployed position, (ii) movement of the retractable cutting apparatus along the incision device, (iii) energy to the incision device, or (iv) movement of one or more stabilizing members associated with said medical device.

16. A medical device for creating elongated incisions within a pericardium, the device comprising:
an incision device operably coupled to a distal end of an elongate catheter, wherein the incision device comprises an incision channel defined along a lateral side of the elongate catheter; and
a retractable cutting apparatus disposed within the incision channel, wherein the retractable cutting apparatus comprises:
a blade structured to move between a retracted position and a deployed position;
a translation body operably coupled to the blade;
a blade rotation pin rotatably connecting the blade to the translation body, wherein the blade rotates about the blade rotation pin; and
an actuation pin attached to a cutting apparatus actuation mechanism structured to move the blade between the retracted position and the deployed position, wherein the actuation pin moves along a blade slot defined on the blade, wherein the movement of the actuation pin along the blade slot causes the blade to move between the retracted position and the deployed position, and
wherein upon movement of the blade into the deployed position, the retractable cutting apparatus is structured to move along the incision channel while the incision device remains stationary.

17. The medical device of claim 16, wherein the blade slot is curved.

18. The medical device of claim 16, further comprising:
a retractable cutting apparatus movement actuator attached to the translation body, wherein the retractable cutting apparatus movement actuator is movable to move the retractable cutting apparatus along the incision channel.

19. A medical device for creating elongated incisions within a pericardium, the device comprising:
an incision device operably coupled to a distal end of an elongate catheter, wherein the incision device comprises an incision channel defined along a lateral side of the elongate catheter;
a retractable cutting apparatus disposed within the incision channel, wherein the retractable cutting apparatus comprises a blade structured to move between a retracted position and a deployed position, wherein upon movement of the blade into the deployed position, the retractable cutting apparatus is structured to move along the incision channel while the incision device remains stationary;
a translation body operably coupled to the blade, wherein the translation body defines a translation body slot;
a blade movement pin attached to the blade, wherein the blade movement pin is positioned to move within the translation body slot, wherein at a first position within the translation body slot, the blade is in the retracted position and at a second position within the translation body slot, the blade is in the deployed position; and
a cutting apparatus casing defining a translation body channel that allows the translation body to move within the translation body channel,
wherein the blade is rotatably attached to the cutting apparatus casing,
wherein the blade is structured to rotate based on the movement of the translation body, and
wherein upon moving the translation body in a first direction, the blade movement pin moves along the translation body slot causing the blade to rotate from a clockwise direction in the retracted position to the deployed position.

20. The medical device of claim 19, further comprising:
a retractable cutting apparatus movement actuator attached to the translation body, wherein the retractable cutting apparatus movement actuator is movable to move the retractable cutting apparatus along the incision channel.
